(19) United States
(12) Patent Application Publication
(10) Pub. No.: US 2004/0241763 A1
(43) Pub. Date: Dec. 2, 2004

(54) Method for diagnosing multiple sclerosis

(76) Inventors: Nir Dotan, Rehovot (IL); Avinoam Dukler, Modeiin (IL); Mikael Schwarz, Kiryat Uno (IL); Ari Gargir, Givatayim (IL)

Correspondence Address: MINTZ, LEVIN, COHN, FERRIS, GLOVSKY, AND POPEO, P.C., ONE FINANCIAL CENTER, BOSTON, MA, 02111, US

(21) Appl. No.: 10/835,607

(22) Filed: Apr. 28, 2004

Related U.S. Application Data
(63) Continuation-in-part of application No. 10/634,309, filed on Aug. 4, 2003.
(60) Provisional application No. 60/400,914, filed on Aug. 2, 2002.
(60) Provisional application No. 60/447,076, filed on Feb. 13, 2003.
(60) Provisional application No. 60/462,984, filed on Apr. 15, 2003.
(60) Provisional application No. 60/473,231, filed on May 23, 2003.

(51) Int. Cl. C12Q 1/68; G01N 33/53; G01N 33/567

(52) U.S. Cl. 435/7.2; 435/6

(57) ABSTRACT

Disclosed is a method for diagnosing multiple sclerosis and more particularly to a method for diagnosing multiple sclerosis by measuring levels of antibodies to glycans in a biological sample.

### RELATED APPLICATIONS

This application claims priority to U.S. Ser. No. 10/634, 309, filed Aug. 4, 2003, which in turn claims priority to U.S. Ser. No. 60/400,914, filed Aug. 2, 2002; U.S. Ser. No. 60/447,076, filed Feb. 13, 2003; U.S. Ser. No. 60/462,984 filed Apr. 15, 2003; and U.S. Ser. No. 60/473,231, filed May 23, 2003. The contents of all of these applications are incorporated herein by reference in their entireties.

### FIELD OF THE INVENTION

The invention relates generally to a method and reagents for diagnosing, and assessing the prognosis of, multiple sclerosis and more particularly to a method and reagents for diagnosing, and assessing the prognosis of, multiple sclerosis by measuring levels of antibodies to glycans in a biological sample.

### BACKGROUND OF THE INVENTION

Multiple sclerosis (MS) is a chronic autoimmune inflammatory disease of the central nervous system. It is a common cause of persistent disability in young adults. In patients suffering from MS, the immune system destroys the myelin sheet of axons in the brain and the spinal chord, causing a variety of neurological pathologies. In the most common form of MS, Relapsing-Remitting, episodes of acute worsening of neurological function (exacerbations, attacks) are followed by partial or complete recovery periods (remissions) that are free of disease progression (stable). It has been reported that ninety percent of patients with MS initially present with a clinically isolated syndrome because of an inflammatory demyelinating lesion in the optic nerve, brain stem, or spinal cord. About thirty percent of those patients with a clinically isolated syndrome progress to clinically definite MS within 12 months of presentation. The subsequent progression of the disease can vary significantly from patient to patient. The progression can range from a benign course to a classic relapsing—remitting, chronic progressive, or rare fulminant course. A method for diagnosing MS that facilitates early MS diagnosis and prediction of disease activity (Benign, Moderate and Malignant) would be valuable for both managing the disease and providing counsel for the patient. For example, patients diagnosed early with active course of MS could be offered disease modifying treatments that have recently been shown to be beneficial in early MS.

Current methods for assessment and tracking progress of MS are based on assessment and scoring of patients' function in attacks and accumulated disabilities during the attacks. One assessment used to assess MS is the Expanded Disability Status Scale (EDSS). However, EDSS score system measures the outcome and does not have predict for the progression of the disease. In addition, EDSS scoring can be variable because it is based on a subjective assessment of patient function. Methods for diagnosis can also include tracking brain lesions by Magnetic Resonance Imaging (MRI) or testing Cerebrospinal Fluid (CSF) for Oligo-Clonal Banding (OCB). MRI is a physical method for assessment of brain lesions and is used widely for MS diagnosis. However, it has only very long term predictive value. In addition, the correlation between MRI results and disease activity is poor. Thus, MRI can not be used for short term projections of disease activity or disease management.

Cerebrospinal puncture is an unpleasant invasive procedure that is not suitable for routine use or prognosis. In addition, both methods assess damage only after it has occurred; neither method can predict the onset of attacks or silent, sub-clinical lesions. A further disadvantage in testing for OCB in CSF and MRI as a way to diagnose MS is that a negative OCB or MRI will not preclude the existence of MS.

Most patients with MS initially present with a clinically isolated syndrome (CIS). Despite the fact that MS will develop in up to 80% of these patients, the course of the disease is unpredictable at its onset. The disease may remain inactive for many years before the appearance of a second clinical relapse or new lesions on MRI confirm the diagnosis. Because currently available therapy is only partially effective and side effects are common, many neurologists are uncertain whether to treat all such patients with immunomodulators, or to wait until the diagnosis is confirmed by a second clinical event or the appearance of new MRI lesions.

There is a need for a simple serological assay that predicts whether patients with a CIS suggestive of MS or newly diagnosed relapsing remitting MS will have a highly active disease course and therefore require aggressive treatment, or whether they will follow a more benign course that enables such patients to postpone immunomodulatory therapy until necessary. This assay would be also useful in helping the diagnosis of MS.

There is also a need for a method that uses objectively assessed markers for diagnosing MS and for predicting disease activity, the onset of attacks or silent lesions in patients suffering from MS.

### SUMMARY OF THE INVENTION

The invention is based in part on the discovery that MS patients have higher serum levels of IgM antibodies that bind the glycan structures Glc(α) or Glc (α, 1-4)Glc(α) or Glc(α, 1-4)Glc(β) as compared to the serum levels of these antibodies in individuals with other neurological diseases (chronic, inflammatory or non-inflammatory), other autoimmune diseases, or healthy individuals. In addition, the same antibodies specific for these glycan structures are found in higher levels for patients in an exacerbation state as compared to the level observed in patients in remission or other subgroups of patients such as Primary Progressive MS (PPMS). The high correlation indicates that the levels of IgM anti Glc(α) or Glc(α, 1-4)Glc(α) or Glc(α, 1-4)Glc(β) in serum can act as a staging and prognostic marker for the activity of the disease. Levels of the antibodies can also be used to decide on treatment and to track the efficacy of treatment.

Measuring the levels of these antibodies in the blood of MS suspected patients facilitates quick and cost effective early diagnosis of MS patients, disease activity prediction and early prescribing of disease modifying drugs. Monitoring of the levels of those antibodies in the blood of defined MS patients also allows for quick and cost effective monitoring of the effects of prescribed drugs, and early detection of attacks or sub-clinical silent lesions, enabling better treatment.

Among the additional advantages of the invention are that the existence of MS in patients can be determined at an earlier stage of the disease, when its symptoms may resemble many other MS-like diseases or when the symptoms are still not sufficient to finally define the patient as having MS. Early diagnosis allows physicians to treat MS earlier in the course of the disease, thereby minimizing or preventing the damage caused by the destruction of myelin and disabilities brought about by this destruction. In addition, the methods disclosed herein enable physicians to follow MS patients regularly in order to assess the disease activity, to monitor therapy, and change treatment once signs for coming attacks appear. For example, an increase in biomarkers indicative of an MS attack may warrant administration to the patient of methylpredisone, which is a general anti inflammatory agent commonly administered during attacks.

In one aspect, the invention features a method of diagnosing, or assessing the prognosis of, multiple sclerosis in a subject. The method includes providing a test sample from a subject and detecting in the test sample at least one biomarker that is an antibody that binds specifically to a glycan structure. The antibody can be, e.g., an anti-Glc(α) antibody, an anti-Glc(α,1-4)Glc(α) antibody, an anti-Glc(α,1-4)Glc(β) antibody, an anti-Glc(β) antibody, an anti-Gal(β) antibody; an anti-Glc(β1-4)Glc(β,1-4)Glc(β) antibody, an anti-GlcNAc(β,1-4) GlcNAc(β) antibody, an anti-L-Araf(α) antibody, an anti-L-Rha(α) antibody, an anti-Gal (β,1-3)[GlcNAc(β,1-6)]GalNAc(α) antibody, an anti-Gal(β,1-4)GlcNAc(α) antibody, an anti-Gal(β,1-3)GalNAc(α), an anti-Gal(β,1-3)GlcNAc(β), an anti-GlcA(β) antibody, or an anti-GlcA(β) antibody, or an anti-Xyl(α) antibody. The levels of antibody or antibodies in the test sample are compared to a control sample, which is derived from one or more individuals who have multiple sclerosis symptoms and have a known multiple sclerosis status, or from an individual or individuals who do not show multiple sclerosis symptoms. MS status can include, e.g., exacerbations, attacks, remissions, benign, moderate, malignant and stable stages of the disease.

In various embodiments, at least 2, 3, 4, 5, 6, 7, 8, 9, 10, 11, 12, 13, 14, 15, 16, 17 or 18 of these antibodies are detected. In some embodiments, the antibody detected in the test sample is an anti-Glc(α) antibody, an anti-Glc(α,1-4)Glc(α) antibody or both an anti-Glc(α) antibody and an anti-Glc(α,1-4)Glc(α) antibody.

In some embodiments, the control sample consists essentially of a population of one or more individuals that do not show symptoms of a multiple sclerosis and do not have multiple sclerosis. In other embodiments, the control sample consists essentially of a population who do show symptoms of a multiple sclerosis and do have multiple sclerosis. In other embodiments, the control sample consists essentially of a population of one or more individuals with neurological diseases other then multiple sclerosis. In other embodiments, the control sample consists essentially of a population of one or more individuals with autoimmune diseases other then multiple sclerosis.

The presence of MS in the control sample can be determined using techniques known in the art, e.g., Clinical neurological examination, or an Expanded Disability Status Scale (EDSS) assessment, Magnetic Resonance Imaging (MRI) assessment, or testing for OCB in the CSF or combination of some or all of the techniques.

The test sample can be, e.g., a biological fluid. Examples of biological fluids include, e.g., whole blood, serum, plasma, spinal cord fluid, urine, or saliva.

The subject can be either a female or a male.

The antibody detected can be, e.g., an IgM type or an IgA type or an IgG antibody.

In some embodiments, the type of multiple sclerosis detected is early multiple sclerosis.

Also provided by the invention is a method of diagnosing a multiple sclerosis exacerbation in a subject. The method includes providing a test sample from a subject and detecting an anti-Glc(α) IgM type antibody and/or an anti-Glc(α, 1-4)Glc(α) IgM type antibody in the test sample. The levels of the antibody in the test sample are compared to a control sample, which is derived from one or more individuals whose multiple sclerosis status is known.

In some embodiments, the control sample consists essentially of a population of one or more individuals that do not show symptoms of a multiple sclerosis exacerbation and whose multiple sclerosis status is in remission. A multiple sclerosis exacerbation is diagnosed in the subject if more anti Glc(α) antibody or anti-Glc(α,1-4)Glc(α) antibody is present in the test sample than in the control sample. In other embodiments, the control sample consists essentially of a population of one or more individuals that show symptoms of a multiple sclerosis exacerbation, and a multiple sclerosis exacerbation is diagnosed in the subject if levels of anti-Glc(α) IgM type antibody and/or anti-Glc(α, 1-4)Glc(α) IgM type antibody are present in similar amounts in the test sample and the control sample.

The test sample can be, e.g., a biological fluid. Examples of biological fluids include, e.g., whole blood, serum, plasma, spinal cord fluid, urine, or saliva.

The subject can be either a female or a male.

The antibody detected can be, e.g., an IgM type or an IgA or an IgG type antibody.

In some embodiments, the diagnosis is an early diagnosis of multiple sclerosis exacerbation.

In some embodiments, the subject has been treated with an MS therapeutic agent, e.g., interferon beta or glitamerer acetate administered subcutaneously.

Also within the invention is method for assessing multiple sclerosis disease activity in a subject. The method includes providing a test sample from a subject and determining whether the test sample contains an anti-Glc(α) IgM type antibody and/or an anti Glc(α, 1-4)Glc(α) IgM type antibody. The amount of antibody in the test sample is compared to the amount of the antibody in the control sample, which is derived from one or more individuals whose multiple sclerosis disease activity is known.

In some embodiments, the control sample consists essentially of a population of one or more individuals whose multiple sclerosis disease activity is defined by Expanded Disability Status Scale (EDSS), changes in an EDSS score, frequency of relapses or a Magnetic Resonance Imaging (MRI) assessment.

The test sample can be, e.g., a biological fluid. Examples of biological fluids include, e.g., whole blood, serum, plasma, spinal cord fluid, urine, or saliva. If desired, the method may further include selecting a therapeutic agent for treating multiple sclerosis by selecting a therapeutic agent and dosage regimen based on the relative levels of the antibody or antibodies in the test sample and the control sample.

In some embodiments, higher levels of antibodies in the test sample relative to the control sample indicate selection of a therapeutic agent and dosage regimen that is subcutaneous administration of interferon beta(BETAFERON®, AVONEX®, REBIF®) or subcutaneous administration of glitamerer acetate (COPAXONE®).

The subject can be either a female or a male.

In a further aspect, the invention provides a method of selecting a therapeutic agent for treating multiple sclerosis. The method includes providing a test sample from a subject diagnosed with, or at risk for, multiple sclerosis and determining whether the test sample contains an anti-Glc(α) antibody. Levels of the antibody in the test sample to are compared to levels of antibody in a control sample consisting essentially of one or more individuals whose multiple sclerosis disease activity is known. A therapeutic agent and dosage regimen is selected based on the relative levels of the antibody in the subject sample and the control sample.

In some embodiments, the method further includes determining whether the test sample contains an anti-Glc((α,1-4)Glc(α)antibody and comparing the levels of the anti-Glc(α,1-4) Glc(α) antibody in the test sample to levels of antibody in a control sample consisting essentially of one or more individuals whose multiple sclerosis disease severity is known.

In some embodiments, the control sample consists essentially of one or more individuals whose status is no multiple sclerosis or stable multiple sclerosis.

In a further aspect, the invention provides a method to predict whether patients with a CIS suggestive of MS or newly diagnosed relapsing remitting MS will have a highly active disease course and therefore require aggressive treatment, or whether they will follow a more benign course that enables such patients to postpone immunomodulatory therapy until necessary.

The method includes providing a test sample from a subject diagnosed with, or at risk for, multiple sclerosis and determining whether the test sample contains an anti-Glc(α, 1-4) Glc(α) antibody. Levels of the antibody in the test sample are compared to levels of antibody in a control sample consisting essentially of one or more individuals whose multiple sclerosis disease activity and course is known. A therapeutic agent and dosage regimen is selected based on the relative levels of the antibody in the subject sample and the control sample.

Also provided by the invention is a kit for diagnosing and predicting disease activity associated with multiple sclerosis. The kit includes a first reagent that specifically detects an anti-Glc(α)antibody, a second reagent that specifically detects an anti-Glc(α, 1-4)Glc(α)antibody, the kit may include one or both reagents, and directions for using the kit. The kit optionally includes a reagent that specifically detects an IgM type antibody.

Also within the invention are substrates that include reagents that specifically detect the antibodies disclosed herein, e.g., an anti-Glc(α) antibody, an anti-Glc((α,1-4)Glc(α) antibody, an anti-Glc(α,1-4)Glc(β) antibody, an anti-Glc(β) antibody, an anti-Gal(β) antibody; an anti-Glc(β, 1-4)Glc(β, 1-4)Glc(β)antibody, an anti-GlcNAc(β, 1-4)GlcNAc(β)antibody, an anti-L-Araf(α) antibody, an anti-L-Rha(α) antibody, an anti-Gal(β,1-3)[GlcNAc(β,1-6)]GalNAc(α) antibody, an anti-Gal(β,1-4)GlcNAc(α) antibody, an anti-Gal(β,1-3)GalNAc(α), an anti-Gal(β,1-3)GlcNAc(β), an anti-GlcA(β) antibody, or an anti-GlcA(β) antibody, or an anti-Xyl(α) antibody. The substrate can be, e.g., planar. In a further aspect, the reagents may be connected to a substrate via a linker.

Also within the invention are reagents, for diagnosing and predicting disease activity associated with multiple sclerosis, that specifically detects an anti-Glc(α,1-4)Glc(α) antibodies and or an anti-Glc(α) antibodies or both. In a further aspect, the reagents may be connected to a substrate via a linker. The substrate may be a bead particles or a planer substrate.

In some embodiment peptides that mimics the specific carbohydrates of this invention can be used for identification of the specific anti glycan antibodies. Unless otherwise defined, all technical and scientific terms used herein have the same meaning as commonly understood by a person of ordinary skill in the art to which this invention belongs. Although methods and materials similar or equivalent to those described herein can be used in the practice or testing of the present invention, suitable methods and materials are described below. All publications, patent applications, patent, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### BRIEF DESCRIPTION OF THE DRAWINGS

FIG. 1 shows the decision tree for determining that a MS suspected patient actually has MS.

FIG. 2 shows the decision tree for selecting a drug and dose for an MS patient based on levels of anti Glc(α, 1-4)Glc(α) or Glc(α) antibodies.

FIG. 3 shows the decision tree for prediction and early diagnosis of attacks in MS patients.

FIG. 4 is a table showing the relative fluorescence from binding of different anti glycan antibodies in MS patients as well as in normal individuals. The glycan structures are presented in the upper line of the table in LINEARCODE® syntax.

FIG. 5 shows the average and median signal for anti glycan antibodies to various glycans from sera extracted from MS patients versus normal control sera. The glycan structures are presented in LINEARCODE® syntax.

FIG. 6 is a graph showing the differences between average signals of MS and healthy individuals. Bars represent a standard deviation. The glycan structures are presented in LIEARCODE® syntax.

FIG. 7A is a graph showing the average signal from binding of anti Glc(α), (Glycan #11) and Glc(α,1-4)Glc(α), (Glycan #12) IgM in MS and healthy populations.

FIG. 7B is a graph showing the average signal from binding of anti Glc (α) Glycan #11 and Glc(α,1-4)Glc(α), (Glycan #12) IgM in MS patients in attack, stable MS patients and healthy populations.

FIG. 8 is a graph showing the correlation between relative fluorescence from adhesion of anti Glc(α) IgM antibodies in anti Glc(α) positive MS patients (left box) negative MS patients (right box) samples and their EDSS levels.

FIG. 9 is a graph showing the temporal stability of the signal from binding of IgM, IgG and IgA anti glycan antibodies over 13 weeks in 7 healthy individuals.

FIGS. 10A-10E show the glycan array; chemical structure, specificity of Lectin interaction and reproducibility. FIG. 10A shows an p-amino phenyl P-saccharide covalently linked at its reducing end to a solid surface via a linker.

FIG. 10B shows batch-to-batch reproducibility of binding of biotinylated WGA to the glycan array. Three separate batches of arrays were assayed simultaneously with biotinylated WGA

FIG. 10C shows a competition assay with ConA to bound Man (α). Increasing concentrations of soluble Mannose or Gal(β,1-4)Glc were incubated with biotinylated ConA (1.5 μg/ml) for 1 hr, and detected with Streptavidin conjugated to Europium.

FIG. 10D shows the specificity of lectin binding to different anomers. ConA binding to negative control Glycerol (19), Man(α) (26) and Man(β) (27). GSI binding to -Gal(α) (1), Gal (β) (2), GalNAc((α) (7), and GalNac(β) (8).

FIG. 10E shows plate-to-plate reproducibility of the glycan array. Five identical plates presenting GlcNac (β) were probed with biotinylated WGA.

FIG. 11A is a histogram showing the binding levels of four serum anti-glycan antibodies after incubation of serum with Glc(α1,4)Glc(α) containing beads (white bars), relative to binding in whole serum (gray bars).

FIG. 11B is a histogram showing the binding profile of antibodies bound to Glc(α1,4)Glc(α) containing beads and eluted with Glc(α1,4)Glc. Black bars—Anti-α-Glc and anti-Ga4Ga antibody levels; Gray bars—Levels of antibody to sugars with Glc moieties; Hatched bars—Levels of antibody to GlcNAc-containing sugars; Empty bars—Levels of antibody to all other sugars presented on the GlycoChipOt substrate.

FIG. 12 is a box plot describing distribution of anti-Glc(α1,4)Glc(α) antibodies in the different groups investigated: RRMS—Relapsing-Remitting MS; PPMS—Primary Progressive MS; OND—Other Neurological diseases; OAD—Other Autoimmune Diseases. The box includes signals of 50% of the population. The line in the box represents the median value. The boundary of the box closest to zero indicates the 25th percentile, and the boundary of the box farthest from zero indicates the 75th percentile. Whiskers above and below the box indicate the 90th and 10th percentiles. Above the box-plots are histograms describing distribution of the population represented by the box and whiskers.

FIGS. 13A and 13B are graphs of the Total IgM and specific anti-Glc(α1,4)Glc(α) antibody levels in MS and control groups. FIG. 13A shows the average level of total IgM in a representative subset of each patient group relative to the level of total IgM in the each group (n=20).

FIG. 13B shows anti-Glc(α1,4)Glc(α) level in a representative subset of each patient group relative to the level of total IgM in the MS group. Both assays were performed on sera diluted to give a signal in the linear range.

FIG. 14A is a box plot describing the distribution of Anti-Glc(α1,4)Glc(α) antibodies in different MS patient groups in relapsing MS (n=41) and remitting MS (n=66) patients. The box includes signals of 50% of the population. The line in the box represents the median value. The boundary of the box closest to zero indicates the 25th percentile, and the boundary of the box farthest from zero indicates the 75th percentile. Whiskers above and below the box indicate the 90th and 10th percentile.

FIG. 14B is a box plot describing the distribution of Anti-Glc(α1,4)Glc(α) antibodies in hyperactive (HA; n=11) and non-hyperactive (non-HA; n=96) RRMS patients. The box includes signals of 50% of the population. The line in the box represents the median value. The boundary of the box closest to zero indicates the 25th percentile, and the boundary of the box farthest from zero indicates the 75th percentile. Whiskers above and below the box indicate the 90th and 10th percentile.

FIG. 14C is a box plot describing the distribution of Anti-Glc(α1,4)Glc(α) antibodies in HA (n=5) and non-HA (n=65) remitting patients. The box includes signals of 50% of the population. The line in the box represents the median value. The boundary of the box closest to zero indicates the 25th percentile, and the boundary of the box farthest from zero indicates the 75th percentile. Whiskers above and below the box indicate the 90th and 10th percentile.

### DETAILED DESCRIPTION OF THE INVENTION

The methods provided herein allow for early diagnosis of initial and recurring multiple sclerosis, as well as prediction of disease activity (Benign, Moderate and Malignant), using objectively assessed biomarker levels.

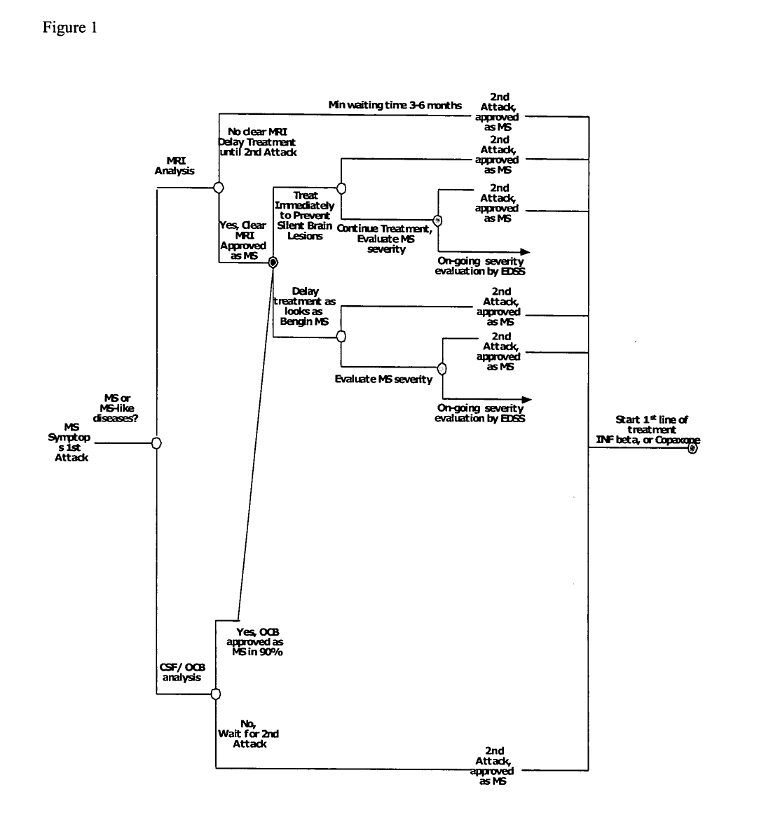

The current decision tree for diagnosing a patient with MS is described in FIG. 1. A patient with acute worsening of neurological function initially has to be diagnosed as a defined MS patient before being eligible for treatment with disease modifying drugs. The physician will have to determine if the patient has MS like symptoms (such as Younger stroke, Lupus, Vitamin B-12 deficiency, Anti phospholipid syndrome, Severe Migraine) or if they actually have MS. The patient will have to experience a second acute worsening of neurological function (attack) before being diagnosed as a MS patient and be able to start chronic treatment with a MS therapeutic agent such as interferon beta or glatiramer acetate.

Currently, physicians are using MRI for the identification of the existence of brain lesions and/or the testing of Cerebrospinal Fluid (CSF) for Oligo Clonal Banding (OCB). If MRI gives a clear result regarding the existence of brain lesions or the presence of OCB in the CSF, the physician may start treatment immediately in order to prevent silent brain lesions. A diagnosis of full MS diagnosis is currently made only after the second attack or the appearance new MRI finding with dissemination in time and space. In case MRI does not give a clear result or there are no OCB in the patients CSF, no MS is diagnosed and treatment is delayed until following a second attack (McDonald et al., Ann Neurol. 50:121-27, 2001.

Most patients with MS initially present with a clinically isolated syndrome (CIS). Despite the fact that MS will develop in up to 80% of these patients, the course of the disease is unpredictable at its onset. The disease may remain inactive for many years before the appearance of a second clinical relapse or new lesions on MRI confirm the diagnosis. Because currently available therapy is only partially effective and side effects are common, many neurologists are uncertain whether to treat all such patients with immunomodulators, or to wait until the diagnosis is confirmed by a second clinical event or the appearance of new MRI lesions. This invention provides a simple serological assay to predict whether patients with a CIS suggestive of MS or newly diagnosed relapsing remitting MS will have a highly active disease course and therefore require aggressive treatment, or whether they will follow a more benign course that enables such patients to postpone immunomodulatory therapy until necessary. This assay is also useful for helping diagnosing MS.

The methods disclosed herein can be performed by extracting blood from a patient with acute worsening of neurological function and suspected to have MS or an already defined RRMS patient. The method can identify the existence of MS and to predict the up coming course of the diseases by measuring anti-Glc(α) and/or anti-Glc(α, 1-4)Glc(α) IgM levels. If the level of at least one of these antibodies is significantly higher then the average level of these antibodies in sera of healthy individuals, patients with neurological diseases other then MS, or patients with autoimmune diseases other then MS, the patient is diagnosed as an MS patient without the need to wait for a second attack or for further MRI findings. In addition, the quick diagnosis allows for treatment to begin immediately.

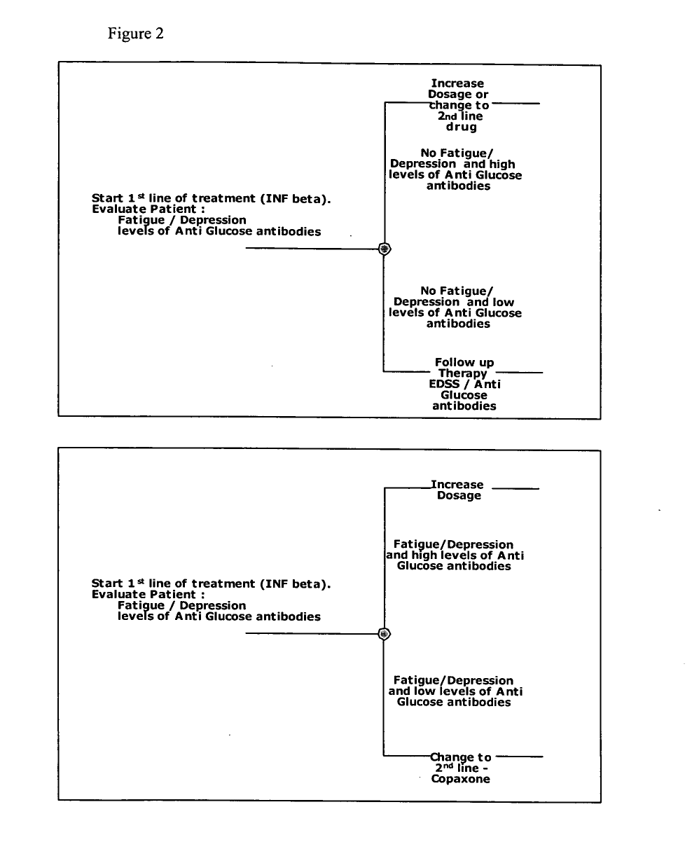

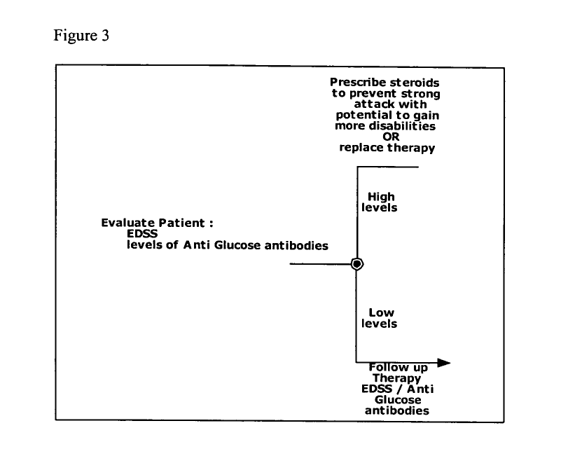

Screening the patient's blood and determining the level of biomarkers disclosed herein, e.g., the IgM antibodies anti Glc(α) and anti Glc(α,1-4)Glc(α) herein allows for accurate monitoring of therapy. For example, one first line of treatment for MS is interferon D (e.g., INFβ-1a and INFβ-1b). The current evaluation of effectiveness and required dosage of the drug is based on continued monitoring of several clinical scores. Currently, the EDSS score and its change over time (e.g., by comparing the difference in the EDSS every 3-6 months) is the main clinical parameter for disease management. An important component of the assessment is the level of fatigue and depression experienced by the patient. The fatigue and or depression can be a symptom of MS, as an autoimmune disease, or a side effect from the usage of interferon beta. Identifying the cause of the fatigue is important for managing the treatment. For example, if the fatigue is a result of a side effect of the interferon, the physician will consider lowering the dosage or even exchanging it for another drug. However, if the fatigue is due to the MS symptoms, the physician will have to consider increasing the drug dosage (see FIG. 2). Significant decreases in antibody levels indicate that the patient is responding well to the given drug.

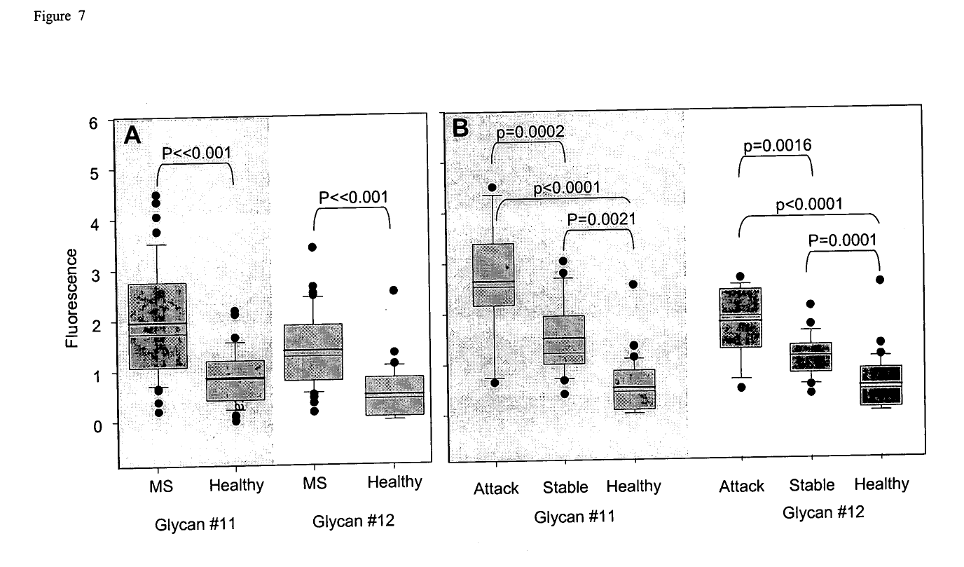

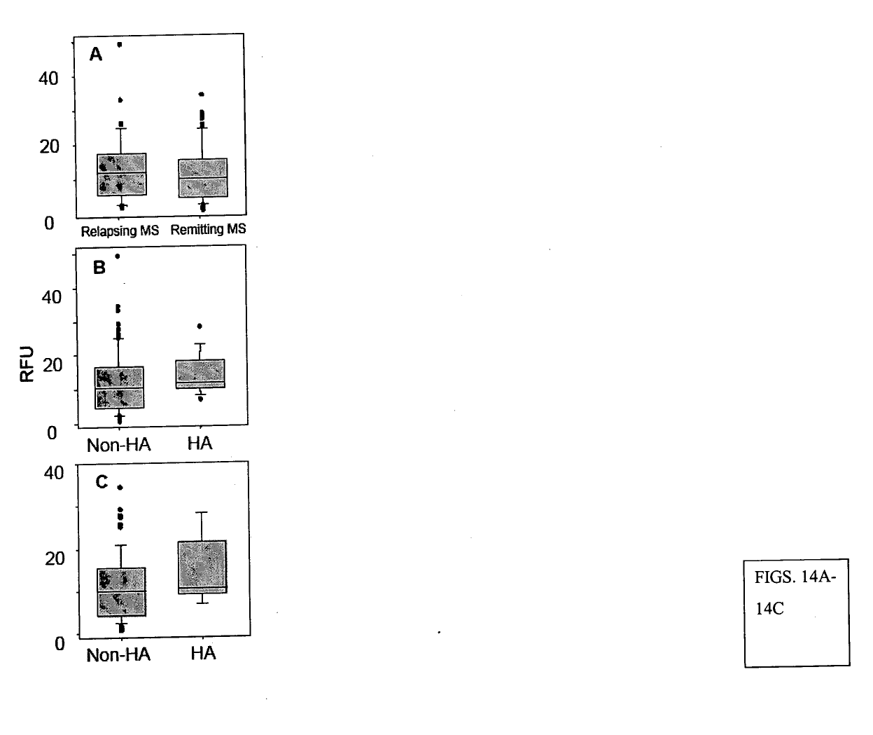

Currently there is no way to predict the onset of attacks and sub-clinical silent lesions in MS patients. MRI and clinical evaluation of the patients can only reveal damage that has already occurred. Periodical measurement of the level of a few anti glycan antibodies (for example anti-Glc(α) IgM or anti-Glc(α,1-4)Glc(α) IgM) in the patient's blood according to the method described herein allows for physicians to predict the upcoming disease activity (e.g. frequency of clinical attacks and appearance of sub clinical MRI silent lesions) based upon an increase in levels of these antibodies. We have found that the levels of these antibodies are higher in the blood of hyper active MS patients then in non hyper active patients (FIG. 14B). We have also found that the levels of these antibodies are significantly higher in the blood of patients in MS attack situations vs. patients in a stable state (see FIG. 7). Upon detection of an increase in those antibodies, the physician can start an aggressive treatment with disease modifying drugs.

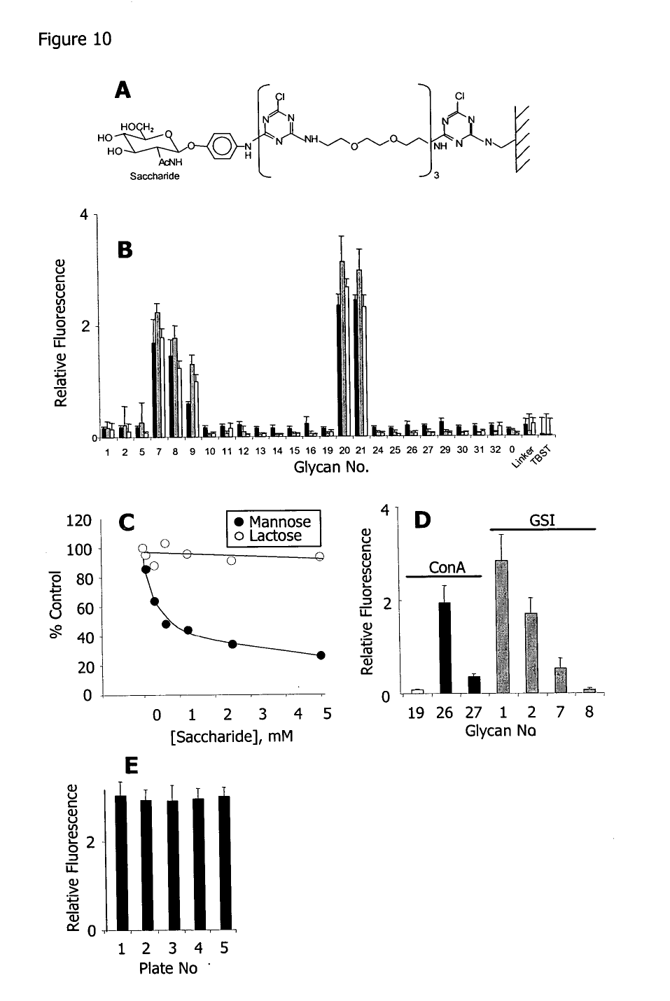

Various glycan structures are discussed in this application. The glycans are presented either in the International Union of Pure and Applied Chemistry (IUPAC) condensed form for nomenclature carbohydrate representation or in LINEARCODE® syntax, for linear code syntax principals see (Banin et al., Glycotechnology 14: 127-137, 2002). Translation of LINEARCODE to IUPAC representation is in Table 1. All the glycan structures that discussed in this disclosure, unless mentioned otherwise are connected to in the indicated anomericity a or P through linker to solid phase as described in FIG. 10A.

In some embodiment peptides that mimics the specific glycans of this invention can be used for identification of the specific anti glycan antibodies. Those peptide that mimics carbohydrate can be identifies for example from screening a filamentous phage-displayed random peptide library (Zhan et al., Biochem Biophys Res Commun. 308:19-22, 2003; Hou et al., J. Immunol. 170:4373-79, 2003).

Most patients with Multiple Sclerosis (MS) initially present with a clinically isolated syndrome (CIS). Despite the fact that clinically definite MS will develop in up to 80% of these patients, the course of the disease is unpredictable at its onset. The disease may remain inactive for many years before the appearance of a second clinical relapse or new lesions on MRI confirm the diagnosis. Because currently available therapy is only partially effective and side effects are common, many neurologists are uncertain whether to treat all such patients with immunomodulators, or to wait until the diagnosis is confirmed by a second clinical event or the appearance of new MRI lesions.

The invention provides a simple serological assay that may be used to predict whether patients with a CIS suggestive of MS or newly diagnosed relapsing remitting MS will have a highly active disease course and therefore require aggressive treatment, or whether they will follow a more benign course that enables such patients to postpone immunomodulatory therapy until necessary.

The invention additionally provides a simple serological test for the definite confirmation of MS and of the level of the risk in individuals presenting a primary acute demyelinating event. Ninety percent of patients with MS initially present with a clinically isolated syndrome due to an inflammatory demyelinating lesion in the optic nerve, brain stem, or spinal cord (O'Riordan et al., Brain 121: 495-503, 1998). Thirty percent of these patients with clinically isolated syndrome will have progression to definite multiple sclerosis within 12 month after presentation (Brex et al,. N. Engl. J. Med. 346:158-164, 2002; O'Riordan et al., Brain 121: 495-503, 1998; Jacobs et al., Ann. Neurol. 41:392-98, 1997), but no more than 80% of patients with a clinically primary event will develop clinically definite MS (Weinshenker et al., Brain 112:1419-28, 1989). Thus, it is desirable to unambiguously confirm and stage MS prior to commencing treatment with disease modifying drugs.

The methods can be used to determine whether a particular treatment MS treatment regimen is warranted for a particular patient. Patients at high risk for rapid progression to definite MS can be offered disease-modifying treatments that have recently been shown to be beneficial in early multiple sclerosis (Comi et al., Lancet 351:1576-82, 2001; Jacobs et al., N. Engl. J. Med. 343:898-904, 2000). On the other hand, for patients at low risk, and who have a chance of remaining relapse free for several years after an initial demyelinating event, immunomodulatory therapy might be postponed until necessary. Thus, an advantage of the invention is better disease management at the early days of the disease.

The invention will be illustrated in the following non-limiting examples.

### EXAMPLE 1

### Comparison Between Antiglycan Antibodies in the Serum of Multiple Sclerosis (MS) Patients and Normal Population

An anti-glycan antibody (Igs) profile was obtained using GlycoChip® arrays (Glycominds, Ltd., Lod, Israel, Cat No. 9100). The arrays were constructed using procedures described in WO00/49412. Anti-glycan antibody profiles of 40 multiple sclerosis patients and 40 sex and aged matched normal blood donors were compared.

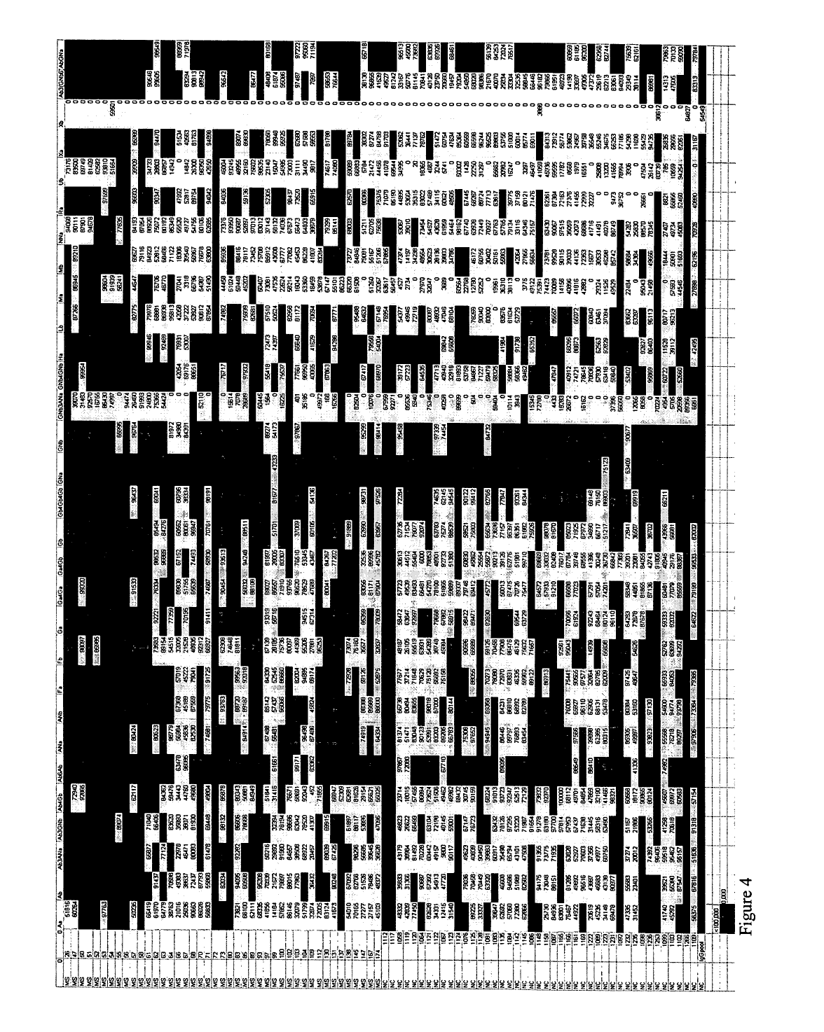

All serum samples were tested using GlycoChip® plates (Glycominds Ltd., Lod, Israel, Cat No. 9100), which was an array of mono and oligosaccharide covalently attached to a reduced volume 384 wells micro titer plate. The mono and oligosaccharides displayed on the array are listed in FIG. 4. A translation of the LinearCode™ syntax used to describe glycan structure to IUPAC nomenclature can be found in Table 1.

The sera of healthy volunteers and MS patients volunteers who had signed an informed consent form were collected in evacuated silicon coated gel containing tubes (Estar Technologies Cat# 616603GLV). The sera were separated from the blood cells and kept frozen in −25° C. until use. They were analyzed in two separate experiments, each repeated twice on separate days.

Sera from volunteers were diluted (1:20) in TBST dispensed into a GlycoChip(9 plate using a Tecan Genesis Workstation 200 robot (10 μL/well) and incubated 30 min at 25° C. There were 4 repeats for each glycan and serum sample on the plate.

The plates were washed with 250 μL/well of high salt buffer (0.15M KNa pH 7.2, NaCl 2M, MgSO₄ 0.085M, 0.05% Tween20) in an automatic plate washer (Tecan, PowerWasher™). Ten μl/well of biotinylated protein A (ICN 62-265), 1 μg/ml in TBST, was dispensed manually and the plates incubated for 30 min at 25° C. The plate was washed again with high salt buffer.

Streptavidin-conjugated Europium, Wallac, AD0062 (1μ/ml, 10 μl/well) was added manually followed by incubation for 30 min at 25° C. in the dark. Washing of the plates with the high salt buffer was repeated. Delfia™ enhancement buffer, (Wallac, 730232, 10 μl/well) was added to the wells and the plates were incubated at least 30 min in the dark. The fluorescence of the wells was read with Victor 1420 (Wallac) using time resolved fluorescence settings Emi. 612 nm and Ext. 340 nmn.

The profiles of all the tested patients are displayed in FIG. 4. The upper 40 lines (MS) describe the anti-carbohydrate level of MS samples, and the lower 40 lines (NC) describe the anti-carbohydrate level of samples from normal control population. The values presented are absolute values without background reduction. Since the detection of bound antibodies was done with biotinylated protein A, which binds to IgG, IgA and IgM., the signal represents the total binding of antibodies from all sub types IgG, IgA and IgM.

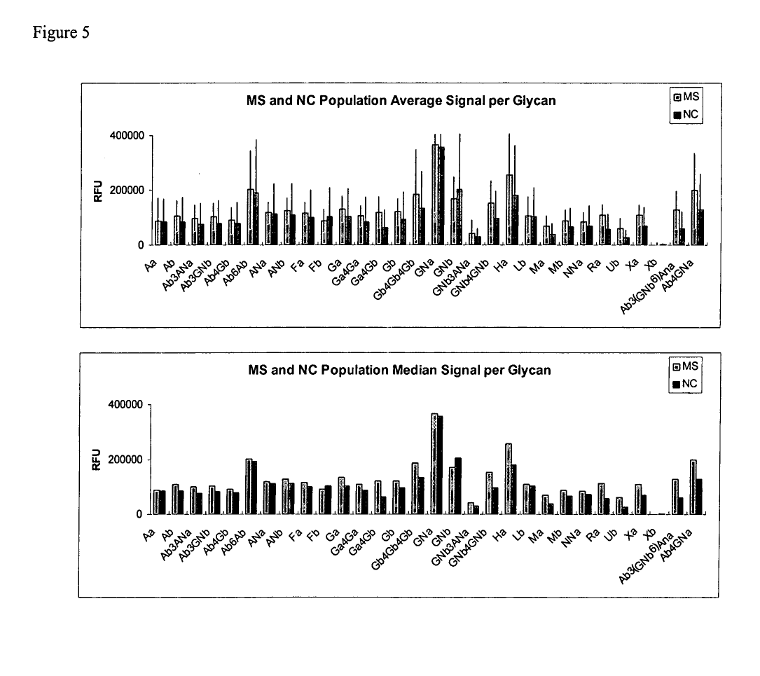

A comparison between the average and median values of anti-carbohydrate antibodies in the MS and normal populations reveals significant differences between the samples from the MS patients and the samples from the normal population, see FIG. 5. One example of a major difference observed between the two groups is the average signal to the glycan Ga4 Gb. A t-test showed that the difference is highly statistically significant (α=0.05; p<0.001). Another example is Ab3(GNb6)ANa, (α=0.05; p<0.001). There are significant differences between the medians of signals of MS and normal population regarding antibodies bound to the following glycans: Glc(α), Glc(α,1-4)Glc(α), Glc(α,1-4)Glc(β), Glc(β), Gal(β), Glc(β,1-4)Glc(β,1-4)Glc(β), GlcNAc (β,1-4)GlcNAc(β), L-Araf(α), L-Rha(α), Gal(β,1-3)[GlcNAc(β,1-6)]GalNAc(α), Gal(β,1-4) GlcNAc(α), Gal(β,1-3)GalNAc(α), Gal(β,1-3)GlcNAc(β), GlcA(β), GlcA(β), Xyl(α). The signal from bound antibodies in MS group is higher then the signal in the normal control group.

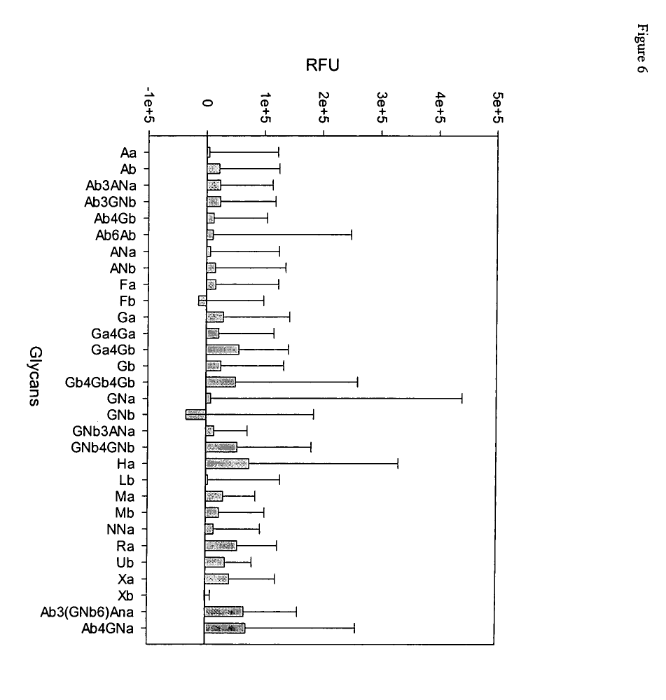

FIG. 6 presents the difference between the average binding values of anti-glycan antibodies between the populations.

### Example 2

### Differences in the Levels of anti-Glc(α), and anti-Glc(α,1-4) Glc(α), a IgM Antibodies in the Serum Between MS Patients in Attack, Stable MS Patients and Healthy Population

A glycan array was used to search for biomarkers among the human serum glycan binding antibody repertoire to differentiate between a healthy population and a group of Multiple Sclerosis (MS) patients, and between MS patients in exacerbation and remission stages. This example demonstrates that two IgM antibodies, anti-Glc(α) and anti-Glc(α,1-4)Glc(α), are found at significantly higher levels in MS patients than in healthy people (sensitivity and specificity of 60% and 93%, respectively), and in MS patients in an exacerbation stage relative to patients in a remission stage (sensitivity and specificity of 89% and 71%, respectively). Also provided is an anti-glycan antibody profile for a healthy population, including a range of variation during a 13 week interval.

The temporal stability of antiglycan antibodies profile over 13 week in apparently healthy individuals was high. The low levels and of anti-Glc(α) and anti-Glc(α,1-4)Glc(α) IgM in a normal population, and their high level in MS patients, and the high temporal stability of anti glycan antibodies suggests that this anti Glc(α) and anti-Glc(α, 1-4)Glc(α) IgM can serve as biomarker for early diagnosis, early prescribing of drugs, monitoring drug effects and prediction of disease activity (e.g. attacks frequency and silent MRI lesions).

All serum samples were tested using GlycoChipg (Glycominds Ltd., Lod, Israel). The glycans were covalently bound to the plastic surface through a linker as previously described (WO02/064556). A list describing the mono- and oligosaccharides tested is provided in Table 1.

Blood samples were obtained from apparently healthy blood donors under an informed consent protocol approved by the Helsinki Human Studies Ethical committees of the Belinson Medical Center in Tel-Aviv, Israel, and Carmel Medical Center in Haifa, Israel. Blood samples were collected from MS patients admitted to the Multiple sclerosis Clinic in Carmel Medical Center in Haifa, Israel. The blood samples were collected in evacuated silicon coated tubes containing gel for the separation of sera from the blood clot (Estar Technologies). After coagulation of the blood, serum was separated by centrifugation and collected. Samples were stored frozen at −25° C. until used.

The volume of all solutions added to the glycan array was 10 μl/well. The sera were diluted (1:20; saturating concentration) in 0.15M Tris-HCl pH 7.2, 0.085M Mg₂SO₄, 0.05% Tween 20 (TBST) containing 1% BSA (Sigma), dispensed into glycan array plates using a Tecan Genesis Workstation 200 automated handling system, and incubated for 60 min at 37° C. The plates were then washed with 250 μL/well Phosphate buffered Saline with 0.05% Tween 20 (PBST, Sigma) in an automatic plate washer (Tecan, PowerWasher™). At this point the following reagents, diluted in TBST with 1% BSA, were added using a Multidrop 384 dispenser (Thermo Labsystems) and incubated for 60 min at 37° C.: for IgG, IgA, and IgM determination—the respective sub-class specific biotinylated goat anti-human Ig antibody (Jackson, Pa., USA) at 2.8 μg/ml, 3 μg/ml, and 0.9 μg/ml, respectively; for total Ig determination—biotinylated Protein A (1 μg/ml, ICN Biomedicals). Following washing with PBST, Streptavidin-conjugated europium (0.1 μg/ml) diluted in TBST with 1% BSA was added to eachwell followed by incubation for 30 min at 37° C. in the dark, and washing with PBST. Delfiarm enhancement solution was then added to the wells and the plates were incubated for 30 to 45 min in the dark at room temperature. The fluorescence of the wells was read with a Victor 1420 (Wallac, Finland) plate reader using time resolved fluorescence settings of 340/612 nm (Excitation/Emission). Differences in the levels of anti-Glc(α) and Glc(α, 1-4)Glc((α) a IgM antibodies in the serum between MS patients in attack, stable MS patients and healthy population.

Serum samples were obtained from MS patients admitted to an outpatient clinic for regular examination after they signed informed consent forms. The patient group was 80% female, approximately reflecting the gender ratio in the general MS population. In accordance with published data (Ritchie et al., J. Clin. Lab. Anal. 12:363-70, 1998), significantly higher levels of IgM (but not IgG or IgA) antibodies were observed in sera from both healthy and MS women as compared to men (not shown). The analysis was therefore limited to the female MS and healthy sub-populations only. Sera of MS patients were initially screened on 54 glycans (Table 1) for the presence of IgG, IgM and IgA anti-glycan antibodies with the purpose of identifying markers that would confirm patients with single acute demyelinating events as MS, and markers that would distinguish between patients during the exacerbation and remission stages of the disease. The experiment was repeated twice using five out of the 54 glycans against which some differences between the groups were found in the initial round.

A reproducible and statistically significant difference in the levels of IgM anti Glc(α) and anti-Glc(α,1-4)Glc(α) antibodies was found between the healthy and MS groups (FIG. 7A), but no significant differences in IgG or IgA levels were found in these studies(not shown). In sera of both groups of MS patients the levels of IgM anti-Glc(α) and anti-Glc((α,1-4)Glc(α) were significantly higher than in the healthy population. An arbitrary set optimal cut-off value (the 97% percentile signal of the “healthy” population) was used to identify positive samples above—and negative samples—below the cut-off value. Thus, anti-Glc(α) binding signals identified correctly 19 out of 42 MS samples (45% sensitivity) and 42 out of 44 apparently healthy sera samples (96% specificity). Measurement of anti-Maltose binding identified correctly 48% of the MS sera and 95% of the apparently healthy sera samples. Defining positive as a sample whose signal is above the cut-off value in either the anti-Glc(α) or Glc(α,1-4)Glc(α) assays improves the sensitivity to 60%, and leaves specificity at 93% (Table 2). The differential distribution of anti-Glc(α) and anti-Glc(α,1-4)Glc(α) antibodies in patients during the exacerbation and remission stages of the disease was significantly higher levels in the former group (FIG. 7B). No difference was found between untreated patients or patients treated with interferon-β (not shown). Using as a cut-off of the 80% percentile of the “stable” MS population, it was determined that anti-Glc(α) binding signals identified correctly 15 out of 18 “attack” samples (83% sensitivity), 19 out of 24 “stable” samples (79% specificity relating to stable as symptom free), and 42 out of 44 “healthy” samples (95% specificity). Measurement of anti-Glc(α, 1-4)Glc(α) binding identified correctly 72% of the attack sera, 79% of the “stable” sera, and 97% of the “healthy sera”. Defining a positive as a sample which signal is above the cut-off value in either the anti-Glc(α) OR anti-Glc(α,1-4)Glc(α) assays, results in sensitivity of 89%, and specificity of 71% and 95% relative to “stable” or “healthy” samples, respectively (Table 3). The high specificity and sensitivity of the anti-Glc(α) and anti-Glc(α,1-4)Glc(α) IgM antibodies make them an efficient tool for early diagnosis and definition of MS patients. The fact that the levels of these antibodies in MS attack situation are much higher then in stable situation make them a tool for early identification and prediction of attacks in relapsing remitting MS patients.

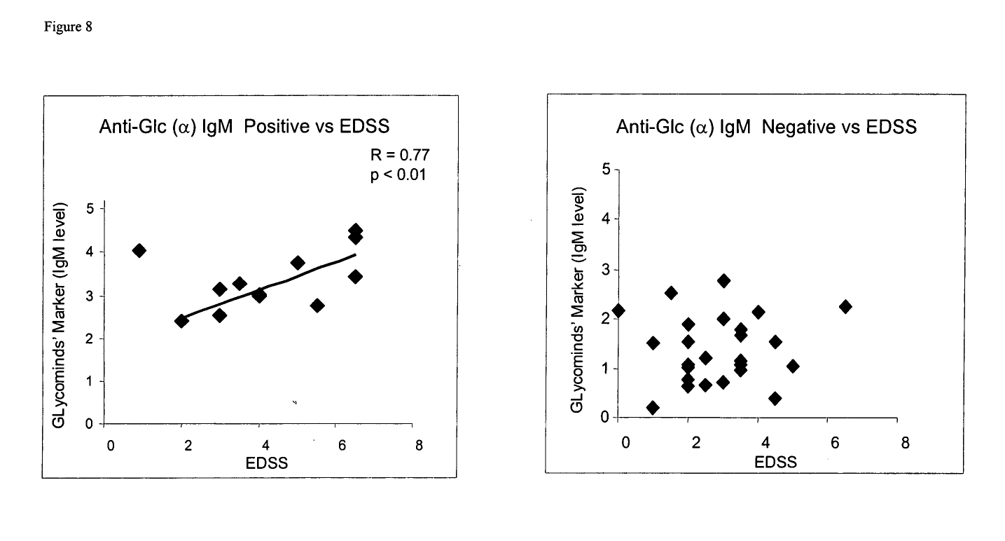

A high correlation between IgM anti-Glc(α) antibody serum levels in female, clinically diagnosed (relapsing-remitting) MS patients, who defined positive for having IgM anti-Glc(α) antibody (as described above), and the women's EDSS (Expanded Disability Status Scale) score was observed, see FIG. 8, left box. There was no correlation between EDSS and the IgM anti-Glc(α) antibody levels in serum for females, clinically diagnosed (relapsing-remitting) MS patients, who defined negative for having IgM anti-Glc(α) antibody, see FIG. 8 left box. The high correlation indicates that the levels of IgM anti-Glc(α) in serum can act as a molecular surrogate biomarker for evaluation the activity of the disease.

Temporal Range of Anti-Glycan Antibody Levels

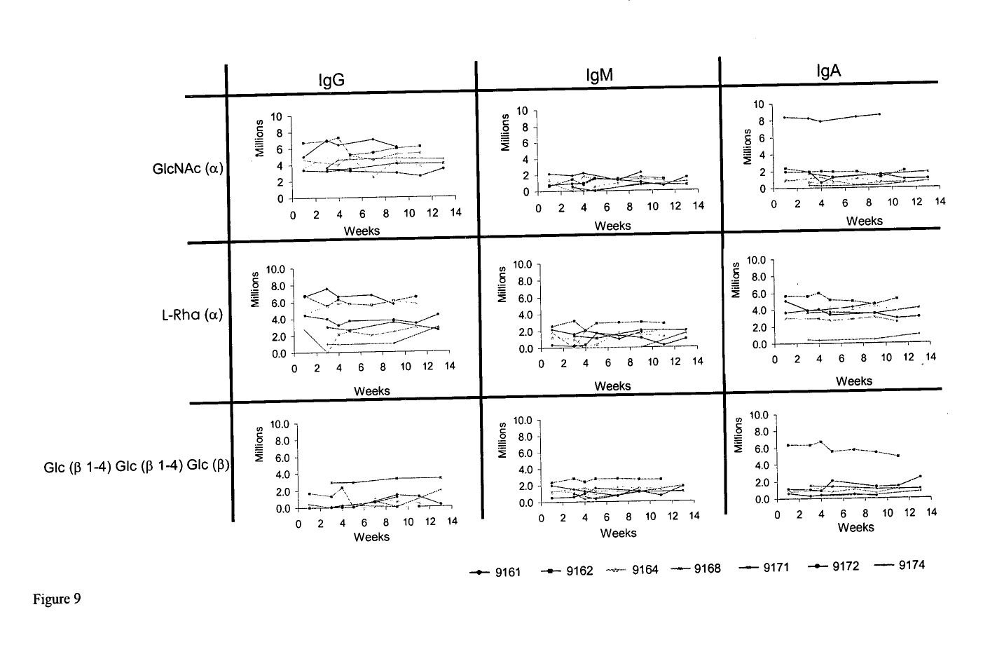

When considering any biological parameter for the use as a surrogate biomarker, it is obviously a prerequisite that the biomarker is not variable in time in the normal population. Thus, the serum levels of IgG, IgA, and IgM anti-L-Rha(α), anti-GlcNAc(α), and -anti Glc(β,1-4)Glc(β,1-4)Glc(β) (β Cellotriose), antibodies in seven healthy volunteers were followed for 13 weeks (FIG. 9). In general, the serum antibody concentrations were found to vary between the different individuals, but to be quite stable over time. For example, sera #9161 and #9162 have extremely high and temporally stable relative levels of IgA anti-GlcNAc(α)and Glc(β,1-4)Glc(β, 1-4)Glc(β) antibodies, respectively, but relatively normal levels of IgA anti L-Rha(α) antibodies and IgG and IgM antibodies. When changes in antibody level do occur they are frequently gradual and continue over several weeks (e.g. serum #9162; IgA anti-Glc(β,1-4)Glc(β,1-4)Glc(α), but can also be sudden, e.g. serum # 9172; IgM anti-L-Rha(α), which suddenly increases between week four and five and then again slowly returns to its basic level.

### Example 3

### Relatively High Levels of IgM Anti-Glc(1,4)Glc(α) Antibodies are Detected in Sera of RRMS Patients and in Hyperactive MS Patients

This example demonstrates the result of screening sera from 107 relapsing-remitting MS (RRMS) patients against a library of glycans on a glycan chip. Significantly higher levels of IgM anti-Glc(α1,4)Glc(α) antibodies were observed in RRMS patients as compared with 77 control patients suffering from other neurological diseases (OND) (p<<0.001) and other autoimmune disease (OAD) (p=0.03) and higher levels than in 20 primary progressive MS patients (PPMS, p=0.06). Slightly higher levels of IgM anti-Glc(α1,4)Glc(α) antibodies were detected in sera from MS patients in relapsing than in remitting state (p=0.11), and in patients with a relatively active disease course (HA) than in non-HA MS patients (p=0.4). The level of anti-Glc(α1,4)Glc(α) antibodies is a specific biomarker for multiple sclerosis and is useful for the clinical prognosis and management of MS.

Sera from outpatients aged 18-55 years with clinically definite and laboratory supported MS, according to the criteria of Poser et al. (Ann. Neurol. 13:227-31, 1983), were eligible for the study. Inclusion criteria for Relapsing-Remitting (RR) patients were: a history of at least two clearly identified and documented relapses in the 2 years prior to study entry, patients were ambulatory, defined by Kurtzke's expanded disability status scale (EDSS) (Kurtze, Neurology 33:1444-52, 1983) of 0-5. Relapse was defined as the appearance or reappearance of one or more neurological abnormalities that persisted for at least 24 hours, and which had been preceded by at least 30 days of stable or improved neurological state. Exclusion criteria were corticosteroids treatment less than 3 months prior to trial, previous immunosuppressive therapy with cytotoxic activity or lymphoid irradiation, as well as pregnancy or lactation. All women were required to use an adequate contraceptive method. Signed informed consent was obtained from patients, and the study was approved by the Ethical Committee of the Lady Davis, Carmel Medical Center and the ethical committee of the Tel-Aviv Sourasky Medical Center.

The 107 patient RRMS group was 76% female, with an average age of 37 years. The different control groups did not significantly differ in gender composition relative to RRMS. The stroke and OND groups were older than the RRMS group. None of the stroke patients were treated with immuno-modulating drugs, while 45% of RRMS patients were untreated and the other 55% of RRMS patients were treated with interferon-β or Glatiramer acetate (Table 4A). Of the OND group (see Table 4B for composition), two patients were under treatment with corticosteroids, and one with anabolic steroids.

Sera of patients suffering from other neurological diseases (OND) and other autoimmune diseases (OAD) were purchased from Genomics Collaborative, USA, or obtained under informed consent from patients admitted to the Neuroimmunology Unit, Carmel Medical Center, Israel and the neurological department of the Tel-Aviv Sourasky Medical Center.

Healthy subjects were included under an informed consent protocol approved by the Helsinki Human Studies Ethical committee of the Rabin Medical Center, Petah Tikva, Israel. The laboratory evaluations were conducted in a blind manner in relation to the clinical findings. The blood samples were collected in evacuated silicon coated tubes containing gel for the separation of sera from the blood clot (Estar Technologies). After coagulation of the blood, serum was separated by centrifugation, collected and kept frozen at −25° C. until use.

All serum samples were tested using a GlycoChip® glycan array (Glycominds Ltd., Lod, Israel). The glycans were covalently bound to the surface through a linker as previously described (WO02/064556; Schwarz et al., Glycobiology 13:749-54, 2003). A list describing the mono-, oligo-, and polysaccharides tested is provided in Table 5.

Fluorescent Assay for Specific Glycan Binding Antibodies Using Multi-Well Plate GlycoChip®.

The volume of all solutions added to the glycan array was 10 μl/well. The sera were diluted 1:40 in 0.15M Tris-HCl pH 7.2, 0.085M Mg₂SO₄, 0.05% Tween 20 (TBST) containing 1% BSA (Sigma), dispensed into glycan array plates using a Tecan Genesis Workstation 200 automated handling system, and incubated for 60 min at 37° C. The plates were then washed with 250 μL/well Phosphate buffered Saline with 0.05% Tween 20 (PBST, Sigma) in an automatic plate washer (Tecan, PowerWasher™). At this point, for IgG, IgA, and IgM determination, the respective sub-class specific biotinylated goat anti-human Ig antibody (Jackson, Pa., USA) diluted in TBST with 1% BSA was added at 2.8 μg/ml, 3 μg/ml, and 0.9 μg/ml, respectively, using a Multidrop 384 dispenser (Thermo Labsystems) and the plate was incubated for 60 min at 37° C. Following washing with PBST, Streptavidin-conjugated Europium (0.1 μg/ml) diluted in TBST with 1% BSA was added to each well followed by incubation for 30 min at 37° C. in the dark, and washing with PBST. Delfia™ enhancement solution was then added to the wells and the plates were incubated for 30 to 45 min in the dark at room temperature. The fluorescence of the wells was read with a Victor 1420 (Wallac, Finland) plate reader using time resolved fluorescence settings of 340/612 nm (Excitation/Emission).

Fluorescent Assay for Specific Glycan Binding Antibodies Using Glass Slide GlycoChip®.

Sera and labeling reagents were incubated on the glass slides in a Tecan HS-4800 hybridization system. Serum samples (130 μl diluted 1:40 in TBST containing 1% BSA) were incubated for 1 hr at 32° C. The samples were circulated and then washed in TNTT buffer (20 mM Tris-HCl, pH7.2; 2M NaCl; 0.05% Tween-20; 0.05% Triton X-100) by the hybridization system. Biotinylated goat anti-human IgM (1:500) and Alexa-488 labeled Streptavidin (1:150; Molecular Probes, USA) were incubated sequentially with washings in-between for 1 hr at 32° C. in the light protected and temperature controlled environment of the hybridization system. Slides were scanned using an Affymetrix 428 array scanner controlled by Jaguar 2.0 (Affymetrix, USA). Images were analyzed using ArrayPro Analyzer 4.5.1.48 (Media Cybernetics, USA).

Statistical Analysis

Descriptive statistics are presented to describe the characteristics of the population andsubgroups. A full factorial model with interactions, predicting antibody level with Age, Gender and Group, resulted with none of the terms emerging as significant. Subsequently, a Main effects model only was used, in which Group emerged significant, while Gender and Age did not. Therefore, based on the two models described, there is no need to adjust for Age and Gender when comparing levels of antibody level between the groups. Q-Q plots showed that antibody level is distributed highly non-normally in some of the groups. A square-root transformation normalized these data, and parametric analyses were conducted on the square-root transformed antibody level data. For comparison between the four disease groups, a one-way ANOVA and Hochberg posthoc procedures were applied. A p value of less than 0.05 was considered to be statistically significant.

Total IgM Fluorescent Assay.

Goat-anti-human IgM antibody (Jackson, Pa., USA; 1 μg/ml in PBS) was adsorbed to the wells of 96 well Maxisorp microtiter plate (Nunc, Denmark) overnight at room temperature. Serial dilutions of sera were added to the wells and incubated for 30 minutes at 27° C. Following washing of the plate in a Powerwasher (Tecan, Switzerland), biotinylated anti-human IgM (Jackson, Pa., USA) was added for 30 min at 27° C. The immobilized immune complex was detected with Streptavidin-Europium as described above.

Specific Glycan Depletion Experiments and Affinity Purification of Anti-Glycan Antibodies.

GlycoFrac™ resin was prepared as described (Bloch et al., FEBS Lett. 44:286-89, 1974). 0.2 ml of 10×TTBS (3 M NaCl, 0.2 M Tris pH 7.8, 1% Tween 20) and 0.11 ml of 4 M NaCl was added to 1.5 ml of MS patient serum pool and the resulting solution was incubated with 1 ml GlycoFrac™ resin in a 15 ml tube for 16 hr at 4° C. with gentle agitation. The serum was collected for glycan binding analysis. The resin was washed extensively with 1×TTBS until the absorbance at 280 nm of the washing solution was below 0.02 O.D. The bound antibodies were eluted in 1 ml batches of soluble glycan (Methyl-D-Glucopyranoside (Sigma, Me-α-Glc) for elution of antibody bound to GlycoFrac™-Glc(α) resin; Glc(α1,4)Glc (Sigma) for elution of antibody bound to (GlycoFrac™-Glc(α1,4)Glc(α), or by 0.2 M glycine, pH 2.8 for elution of antibody bound to GlycoFrac™-Rha(α). The pooled fractions were dialyzed over-night against PBS containing 0.02% Sodium Azide.

Screening for Glycan Structure Antigens Differentiating Between Sera from MS Patients and Control Patients.

Sera of 66 RRMS patients and 13 healthy subjects were initially screened on a microtiter well GlycoChip® containing a library of 40 different glycans for the presence of IgG, IgM and IgA anti-glycan antibodies, with the purpose of identifying markers that would distinguish between MS and control samples. Several IgM antibodies—of which anti-Glc(α) and anti-Glc(α1,4)Glc(α) where the most prominent—were found to discern between sera from RRMS patients and healthy individuals (Table 6). No differences were observed for IgG and IgA antibodies in these studies (not shown).

Specificity Screening

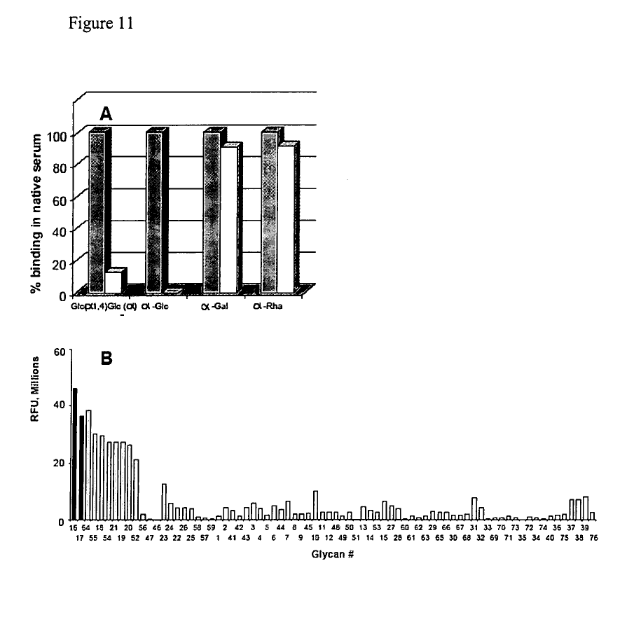

In order to demonstrate that the antibody binding observed is specific for the attached sugars, a depletion experiment was performed in which an MS patient sera pool was pre-incubated with GlycoFrac™ resin containing specific glycans. FIG. 11A demonstrates that IgM antibodies specific to Glc(α1,4)Glc(α) and to Glc(α) are practically depleted from the serum after incubation with the Glc(α1,4)Glc(α)-beads, while binding to Gal(α) and Rha(α) remains intact. The antibodies bound to the Glc(α1,4)Glc(α)-beads were specifically eluted with Glc(α1,4)Glc and applied to a GlycoChipg. FIG. 11B shows that these affinity purified antibodies react specifically with glucose based structures such as Glc(α1,4)Glc(α) and Glc(α) (glycans number 16 and 17), and other sugars containing free glucose residues (glycan numbers 18-21, 52, 54, 55). Interestingly, the affinity purified antibodies bound to LPS from S. typhimurium, which contains Glc (α,1-4) linkages (Lind S, Lindberg AA. Epitope size, specificity and equilibrium constant for four monoclonal antibodies binding to the 0:4 polysaccharide antigen of Salmonella serogroup B bacteria. Mol Immunol. 1992;29:1013-1023 and Holme T, Lindberg AA. Structural studies on the O-specific side-chains of the cell-wall lipopolysaccharide from Salmonella typhimurium 395 ms. Carbohydrate Research. 1968;8:43-55) but not LPS molecules lacking Glc(α1,4) derived from 4 other bacterial strains. All glycans presented on the GlycoChip® different from or lacking Glc-residues were not recognized by the affinity purified antibodies (e.g. Gal(α), GlcNAc(β,1-4) GlcNAc(β), Rha(α), glycan numbers 2, 26, 27). The specificity of anti-Glc(α) and anti-GlcNAc(α) was similarly analyzed and were found to be ligand specific (not shown). These results show that the binding measured is specific for the indicated glycans.

IgM Anti-Glycan Antibody Levels in RRMS and Control Populations

The results of the pilot experiment described above in which significant differences in levels of IgM anti-Glc(α,1-4)Glc(α), and anti-Glc(α) were observed between RRMS and healthy controls, together with the certainty that the binding assay is specific, prompted as to test if the phenomenon of elevated levels of these antibodies is specific to RRMS patients among patients with similar neurological symptoms or other autoimmune diseases (see Table 7B). It was also addressed whether a correlation exists between the activity of the disease and the level of the markers. In order to ensure that the results of the pilot experiment were not platform dependent, the next set of studies were performed on the glass slide based GlycoChip®, and not on plastic multiwell plates.

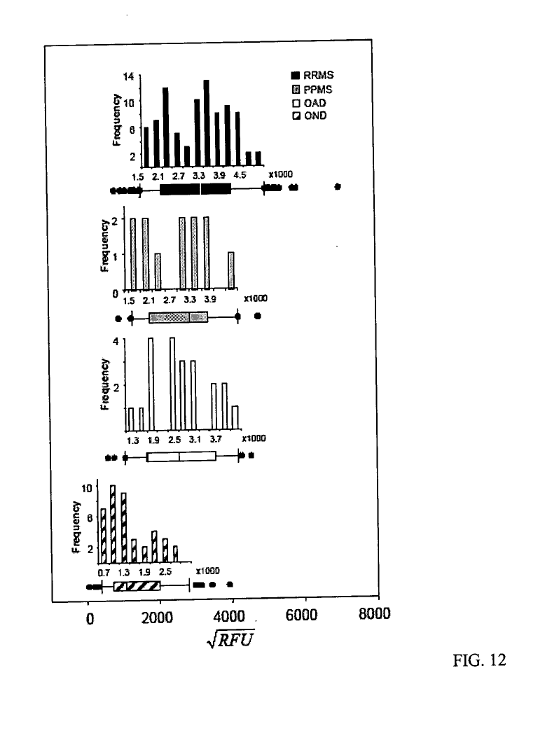

Significantly higher levels of IgM anti-Glc(α,1-4)Glc(α) were detected in RRMS as compared with sera from patients with OND (p<<0.001), or OAD (p=0.03). Higher levels IgM anti-Glc(α,1-4)Glc(α) were also observed relative to levels detected in sera from Primary Progressive MS (PPMS, p=0.06). Anti-Glc(α) and anti-Rha(α) antibodies were significantly higher in RRMS than in OND (p<<0.001), but were similar to the levels in OAD and PPMS. Significantly higher levels of anti-GlcNAc(α) antibodies were recorded in RRMS as compared with OND (p<<0.001) and OAD (p=0.02), but not as compared with PPMS (p=0.3) (FIG. 12 and Table 8). The results suggest that the anti-Glc(α,1-4)Glc(α) antibody diverges significantly between RRMS and all other control groups and is a strong candidate for an IgM MS biomarker.

Total IgM

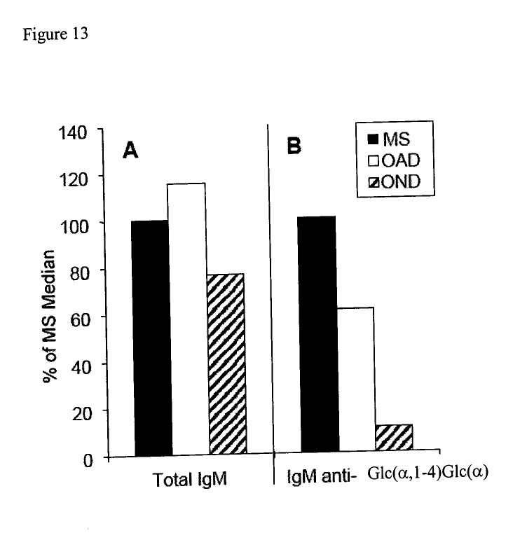

The levels of anti-glycan IgM antibodies measured in the OND group were the lowest among the patient groups tested. A test measuring total IgM in the different groups showed that this group was not suppressed in general; while the concentration of total IgM in OND was about 70% of that measured in MS, the specific anti-Glc(α, 1-4)Glc(α) level in OND was only 11% of that in MS (FIG. 13).

Anti-Glc(α, 1-4)Glc(α) Levels in Relapsing and Remitting RRMS

Anti-Glc(α,1-4)Glc(x) levels were somewhat (p=0.14), but consistently, higher in relapsing as compared with remitting MS patients. (FIG. 14A). A possible reason for the relatively small difference between the relapsing and remitting groups is that patients in remission were having sub-clinical events that may be the reason for high levels of anti-Glc(α, 1-4)Glc(α). Therefore, further stratification of the RRMS population was done; “Hyperactive” (HA) patients were defined as either having three or more relapses per year in the last two consequent years or an EDSS change of two points per year in the last two consequent years. FIGS. 14B and 14C show that the HA patients have a higher level of antibodies in comparison to other patients. This may suggest that these anti-glycan antibodies can serve as markers for disease progression.

The difference in specific antibody level of the different groups is not the result of a general suppression of IgM production in OND, since the total IgM antibody level of the OND group relative to the total IgM level in RRMS is much higher than the anti-Glc(α, 1-4)Glc(α) level of the OND group relative to RRMS (FIG. 13). This suggests that there is specific regulation of the production of these antibodies, which is modulated differentially in the different neurological diseases.

The presence of higher serum antibodies in RRMS could be a result of the body's attempt to repair the damaged myelin sheath similarly to human monoclonal antibodies (directed against oligodendrocyte surface molecules such as the carbohydrate epitope HNK-1) that promote remyelination (Bieber et al., Glia 37:241-49, 2002), or it could be part of the immune systems assault on the myelin sheath (von Budingen et al., Proc. Nat. Acad. Sci. (USA) 99:8207-12, 2002, similarly to other well characterized neurological autoimmune diseases in which surface molecules in synapses are targeted by autoantibodies (Lang et al., Autoimmun. Rev.: 2:94-100, 2003). The finding of the anti-Glc(α,1-4)Glc(α) antibody in RRMS patients is fascinating in light of reports of the presence of amylolytic activity of IgM and IgG antibodies in sera from MS patients (Saveliev et al., Immunol. Lett. 86:291-97, 2003), but not in sera from healthy individuals. Also, it was suggested that the specific autoantibody recognition of an N-glycosylated MOG peptide is most likely driven by direct interactions of the antibody binding site with the Asn-linked Glc(β) moiety (Carotenuto et al., J. Med. Chem. 44:2378-2381, 2001; Mazzucco et al., Bioorg. Med. Chem. Lett. 9:167-72, 1999). This interesting finding corroborates the idea that glucose-containing structures are involved in the MS pathology. However, the antigen described by Mazzucco et al. is different from the glycans recognized by the antibodies in the present invention, since the glycopeptide as a whole was shown to be the epitope, while the sugar moiety alone was sufficient to specifically bind antibodies present in MS sera. Together, these findings suggest an important role for sugars in the pathogenesis of MS.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

Tables and Figures:

TABLE 1
Saccharides displayed on the glycan array

| Glycan | IUPAC | LINEARCODE ® | Common Name |
|---|---|---|---|
| 0 | pNP-OH | pNP-0 | |
| 1 | Gal (α) | Aa | |
| 2 | Gal (β) | Ab | |
| 3 | Gal (β 1-3) GalNAc | Ab3ANa | |
| | (α) | | |
| 4 | Gal (β 1-3) GlcNAc | Ab3GNb | |
| | (β) | | |
| 5 | Gal (β 1-4) Glc (β) | Ab4Gb | Lactose |
| 6 | Gal (β 1-6) Gal (β) | Ab6Ab | |
| 7 | GalNAc (α) | ANa | |
| 8 | GalNAc (β) | ANb | |
| 9 | Fuc (α) | Fa | |
| 10 | Fuc (β) | Fb | |
| 11 | Glc (α) | Ga | |
| 12 | Glc (α 1-4) Glc (α) | Ga4Ga | Maltose |
| 13 | Glc (α 1-4) Glc (β) | Ga4Gb | |
| 14 | Glc (β) | Gb | |
| 15 | Glc (β 1-4) Glc (β) | Gb4Gb | Cellobiose |
| 16 | Glc (β 1-4) Glc | Gb4Gb4Gb | Cellotriose |
| | (β 1-4) Glc (β) | | |
| 17 | Glc (β 1-4) Glc | Gb4Gb4GbGb4Gb | Cellopentaose |
| | (β 1-4) Glc (β 1-4) | | |
| | Glc | | |
| 18 | Glycerol | Glycerol | |
| 19 | GlcNAc (α) | GNa | |
| 20 | GlcNAc (β) | GNb | |
| 21 | GlcNAc (β 1-3) | GNb3ANa | |
| | GalNAc (α) | | |
| 22 | GlcNAc (β 1-4) | GNb4GNb | Chitobiose |
| | GlcNAc (β) | | |
| 23 | L-Rha (α) | Ha | |
| 24 | GalA (β) | Lb | |
| 25 | Man (α) | Ma | |
| 26 | Man (β) | Mb | |
| 27 | Neu5Ac (α) | NNa | |
| 28 | L-Araf (α) | Ra | |
| 29 | GlcA (β) | Ub | |
| 30 | X(α) | Xa | |
| 31 | X(β) | Xb | |
| 32 | Gal (β1-3) [GlcNAc | Ab3(GNb6)ANa | |
| | (β1-6)] GlcNAc (β) | | |
| 33 | Gal (β 1-4) | Ab4GNa | |
| | GlcNAc (α) | | |
| 34 | Gal (α1-3) Gal | Aa3Ab4GNb | Linear B-2 |
| | (β 1-4) GlcNAc (β) | | |
| 35 | Gal (β1-3) Gal | Ab4GNb | N-Acetyl |
| | (β 1-4) GalNAc (β) | | |
| 36 | Man (β1-4) | Mb4Gb | |
| | GlcNAc (β) | | |
| 37 | GlcNAc (β1-6) | GNb6ANa | |
| | GalNAc (α) | | |
| 38 | Fuc (α 1-2) | Fa2Ab | |
| | Gal (β) | | |
| 39 | Neu5Ac (α2-3) Gal | NNa3Ab4(Fa3)GNb | Sialyl Lewis X |
| | (β 1-4) [Fuc (α 1- | | |
| 40 | Man (α 1-3) Man (α) | Ma3Ma | |
| 41 | GlcNAc (β) 6-sulfate | GN[6S]b | |
| 42 | Glc (β 1-3) Glc (β) | Gb3Gb | |
| 43 | Gal(β) 3-sulfate | A[3S]b | |
| 44 | Neu5Ac (α1-3) Gal | NNa3Ab4GNb | Sialyl lactosamine |
| | (β 1-4) GlcNAc (β) | | |
| 45 | Man (α 1-3) [Man | Ma3(Ma6)Mb | |
| | (α1-6)] Man (β) | | |
| 46 | Neu5Ac (α1-3) Gal | NNa3Ab4Gb | Sialyl lactose |
| | (β 1-4) Glc (β) | | |
| 47 | GlcNAc (β 1-3) Gal | GNb3Ab4Gb | Lacto-3 |
| | (α1-4) Glc (β) | | |
| 48 | Gal (α1-4) Gal | Aa4Ab4Gb | Pk antigen |
| | (β 1-4) Glc (β) | | |
| 49 | Neu5Ac (α1-6) Gal | NNa6Ab4GNb | |
| | (β 1-4) GlcNAc (β) | | |
| 50 | Gal (a 1-4) [Fucp | Ab4(Fa3)GNb | Lewis X |
| | (a 1-3)]GlcNAc (b) | | |
| 51 | Neu5Ac (α1-3) Gal | NNa3Ab3(Fa4)GNb | Sialyl Lewis A |
| | (β 1-4) [Fuc (α1-3] | | |
| 52 | Man (α 1-6) | Ma6Ma | |
| | Man α | | |
| 53 | Neu5Ac (α1-3) Gal | NNa3Ab3GNb | Sialyl Lewis c |
| | (β 1-3) GlcNAc (β) | | |
| 54 | Neu5Ac (α1-3) Gal | NNa3Ab3ANa | SiT antigen |
| | (β 1-3) GalNAc (α) | | |

TABLE 2
Number of positive samples having binding signals above
the 97% percentile of healthy population.

| Glycan | Result | MS | Healthy |
|---|---|---|---|
| Glc (α) | Positive | 19/42 (45%) | 2/44 (4.5%) |
| | Negative | 23/42 (55%) | 42/44 (96%) |
| Glc (α 1-4) Glc | Positive | 20/42 (48%) | 2/44 (4.5%) |
| (α) | Negative | 22/42 (52%) | 42/44 (96%) |
| Glc (α 1-4) Glc | Positive | 25/42 (60%) | 3/44 (6.8%) |
| (α) OR Glc (α) | Negative | 17/42 (40%) | 41/44 (93%) |

TABLE 3
Number of positive samples having binding signals above
the 80% percentile of “stable” MS population.

| Glycan | Result | Attack | Stable | Healthy |
|---|---|---|---|---|
| Glc (α) | Positive | 15/18 (83%) | 5/24 (21%) | 2/44 (4.5%) |
| | Negative | 3/18 (17%) | 19/24 (79%) | 42/44 (96%) |
| Glc (α 1-4) Glc | Positive | 13/18 (72%) | 5/24 (21%) | 2/44 (4.5%) |
| (α) | Negative | 5/18 (28%) | 19/24 (79%) | 42/44 (96%) |
| Glc (α 1-4) Glc | Positive | 16/18 (89%) | 7/24 (29%) | 2/44 (4.5%) |
| (α) OR Glc (α) | Negative | 2/18 (11%) | 17/24 (71%) | 42/44 (96%) |

TABLE 4A
Patient Characteristics

| | | Relapsing | Remitting | | | | | | | | |
|---|---|---|---|---|---|---|---|---|---|---|---|
| | RRMS | MS | MS | PPMS | Stroke | OND | ONIND | OIND | OAD | RA | Chron's |
| N | 107 | 41 | 66 | 16 | 9 | 50 | 30 | 11 | 27 | 15 | 12 |
| Age, mean (SD), | 37 (11) | 35 (10) | 38 (11) | 50 (10) | 59 (20)* | 55 (13)* | 56 (11) | 49 (7) | 40 (10) | 44 (9) | 37 (10) |
| years | | | | | | | | | | | |
| Women, n (%) | 81 (76) | 32 (78) | 49 (74) | 9 (56) | 3 (33) | 22 (44) | 14 (47) | 5 (45) | 21 (76) | 12 (77) | 9 (75) |
| Medication | | | | | | | | | | | |
| Copaxone ®, | 15 (14) | 4 (10) | 11 (17) | — | — | — | — | — | — | — | — |
| n (%) | | | | | | | | | | | |
| Betaferon ®, | 10 (9) | 5 (12) | 5 (8) | — | — | — | — | — | — | — | — |
| n (%) | | | | | | | | | | | |
| Rebif ®, n (%) | 16 (15) | 6 (15) | 10 (15) | — | — | — | — | — | — | — | — |
| Avonex ®, n (%) | 17 (16) | 6 (15) | 11 (17) | — | — | — | — | — | — | — | — |
| Total INF-β, | 43 (40) | 17 (41) | 26 (39) | — | — | — | — | — | — | — | — |
| n (%) | | | | | | | | | | | |
| Total treated, | 58 (54) | 21 (51) | 37 (56) | — | | | | | | | |
| n (%) | | | | | | | | | | | |
| Untreated, n (%) | 49 (46) | 20 (49) | 29 (44) | 16 (100) | | | | | | | |

MS, multiple sclerosis;
OAD, other autoimmune diseases;
OIND, other inflammatory neurological diseases;
OND, other neurological diseases;
ONIND, other non-inflammatory neurological diseases;
PPMS, primary progressive multiple sclerosis;
RA, Rheumatoid arthritis;
RRMS, relapsing-remitting multiple sclerosis.

TABLE 4B
Make-Up of the OND Group

| | No. of cases |
|---|---|
| Chronic inflammatory (OIND) | |
| Meningitis | 4 |
| Guillian-Barre syndrome | 1 |
| Myasthenia gravis | 1 |
| Encephalitis | 1 |
| Chronic non-inflammatory (ONIND) | |
| Parkinson's disease | 3 |
| Facialis | 1 |
| Movement disorder | 2 |
| Dystonia | 1 |
| Migraine headache | 8 |
| Meniere's disease | 2 |
| ALS | 1 |
| Huntington's disease | 1 |
| Acute | |
| Stroke | 9 |

TABLE 5
Saccharides displayed on the glycan arrays^{a}

| Glycan | | |
|---|---|---|
| number | IUPAC nomenclature | LinearCode ®^{b} |
| 1 | Gal(β) 3-sulfate | A[3S]b |
| 2 | Gal(α) | Aa |
| 3 | Gal(β) | Ab |
| 4 | Gal(β1-3)[GlcNAc(β1-6)]GalNAc(α) | Ab3(GNb6)ANa |
| 5 | Gal(β1-3)GalNAc(α) | Ab3ANa |
| 6 | Gal(β1-3)GlcNAc(β) | Ab3GNb |
| 7 | Ga1(β1-4)Glc(β) | Ab4Gb |
| 8 | Gal(β1-4)GlcNAc(α) | Ab4GNa |
| 9 | Gal(β1-3)Gal(β1-4)GalNAc(β) | Ab4GNb |
| 10 | Gal(β1-6)Gal(β) | Ab6Ab |
| 11 | GalNAc(α) | ANa |
| 12 | GalNAc(β) | ANb |
| 13 | Fuc(α) | Fa |
| 14 | L-Fucp(α1-2)D-Galp(β) | Fa2Ab |
| 15 | Fuc(β) | Fb |
| 16 | Glc(α) | Ga |
| 17 | Glc(α1-4)Glc(α) | Ga4Ga |
| 18 | Glc(α1-4)Glc(β) | Ga4Gb |
| 19 | Glc(β) | Gb |
| 20 | Glc(β1-3)Glc(β) | Gb3Gb |
| 21 | Glc(β1-4)Glc(1-4)Glc(β) | Gb4Gb4Gb |
| 22 | GlcNAc(β) 6-sulfate | GN[6S]b |
| 23 | GlcNAc(α) | GNa |
| 24 | GlcNAc(β) | GNb |
| 25 | GlcNAc(β1-3)GalNAc(α) | GNb3ANa |
| 26 | GlcNAc(β1-4)GlcNAc(β) | GNb4GNb |
| 27 | L-Rha(α) | Ha |
| 28 | GalA(β) | Lb |
| 29 | Man(α) | Ma |
| 30 | D-Manp(α1-3)D-Manp(α) | Ma3Ma |
| 31 | Man(β) | Mb |
| 32 | D-Manp(β1-4)D-Glcp(β) | Mb4Gb |
| 33 | Neu5Ac(α) | NNa |
| 34 | Neu5Ac(α1-3)Gal(β1-4)GlcNAc(β) | NNa3Ab4GNb |
| 35 | D-Neup5Ac(α2-3)D-Galp(β1-4)[L-Fucp (α1-3)]D- | NNa3Ab4(Fa3)GNb |
| | GlcpNAc(β) | |
| 36 | L-Araf(α) | Ra |
| 37 | GlcA(β) | Ub |
| 38 | X(α) | Xa |
| 39 | X(β) | Xb |
| 40 | PNP | pNP |
| 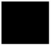 | 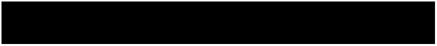 | 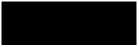 |
| 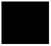 | 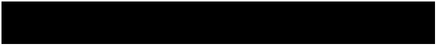 | 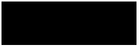 |
| 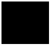 | 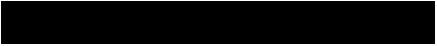 | 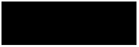 |
| 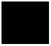 | 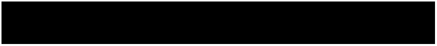 | 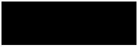 |
| 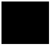 | 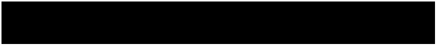 | 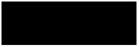 |
| 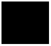 | 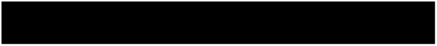 | 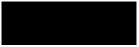 |
| 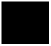 | 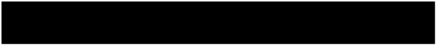 | 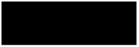 |
| 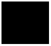 | 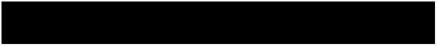 | 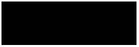 |
| 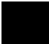 | 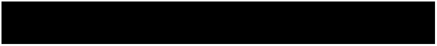 | 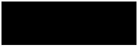 |
| 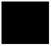 | 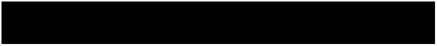 | 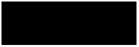 |
| 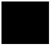 | 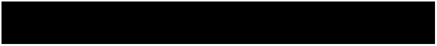 | 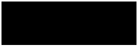 |
| 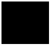 | 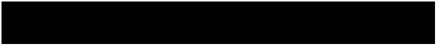 | 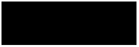 |
| 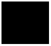 | 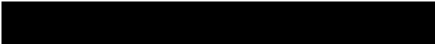 | 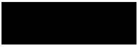 |
| 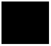 | 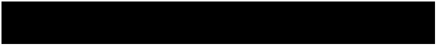 | 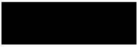 |
| 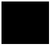 | 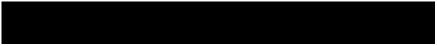 | 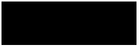 |
| 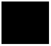 | 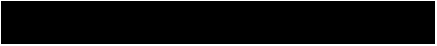 | 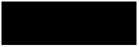 |
| 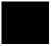 | 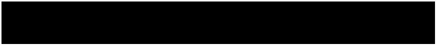 | 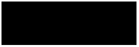 |
| 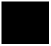 | 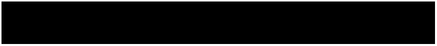 | 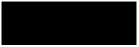 |
| 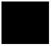 | 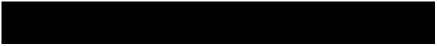 | 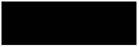 |
| 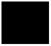 | 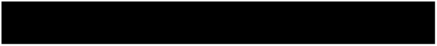 | 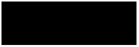 |
| 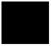 | 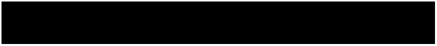 | 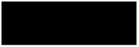 |
| 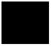 | 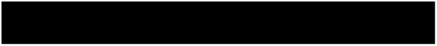 | 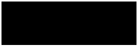 |
| 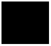 | 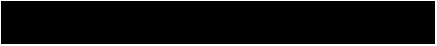 | 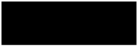 |
| 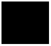 | 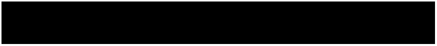 | 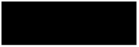 |
| 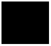 | 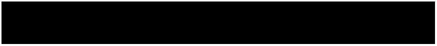 | 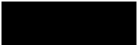 |
| 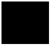 | 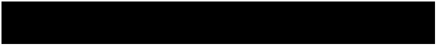 | 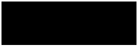 |
| 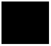 | 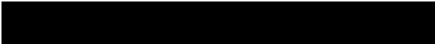 | 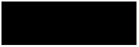 |
| 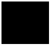 | 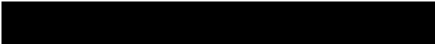 | 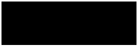 |
| 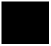 | 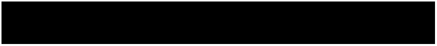 | 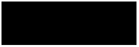 |
| 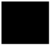 | 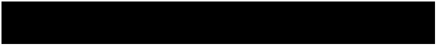 | 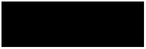 |
| 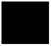 | 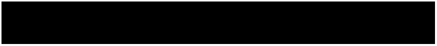 | 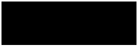 |
| 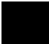 | 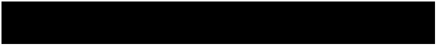 | 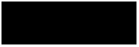 |
| 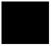 | 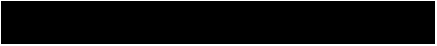 | 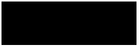 |
| 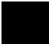 | 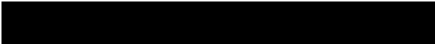 | 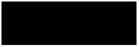 |
| 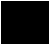 | 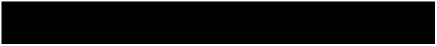 | 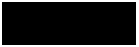 |
| 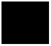 | 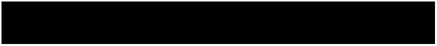 | 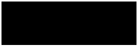 |

^{a}All ligands in the table were used in the specificity screen described in FIG. 1. The ligands in the non shaded cells were used in the discovery screening.
^{b}A syntax that describes the branched structure of glycans in a linear computer-friendly mathematical formula.
⁵⁰ALS, Amyotrophic lateral sclerosis; OIND, other inflammatory neurological diseases; OND, other neurological diseases; ONIND, other non-inflammatory neurological diseases.

TABLE 6
Descriptive Statistics for anti-glycan antibody levels in RRMS and control groups

| Sugar Ligand | Signal | RRMS | Relapsing MS | Remitting MS | PPMS | OAD | OND |
|---|---|---|---|---|---|---|---|
| Glc(α1,4)Glc(α) | Median, RFU | 10,583,221 | 11,965,877 | 9,903,854 | 8,237,277 | 6,473,243 | 1,146,109 |
| | SD | 8,769,550 | 9,609,488 | 8,127,044 | 6,412,262 | 6,058,448 | 6,560,037 |
| | p-value (vs RRMS) | | | | 0.25 | 0.06 | >>0.001 |
| α-Glc | Median, RFU | 8,919,418 | 11,138,222 | 8,473,512 | 6,533,652 | 6,659,457 | 2,079,786 |
| | SD | 9,880,138 | 10,902,872 | 9,082,252 | 13,404,687 | 6,281,151 | 5,626,853 |
| | p-value (vs RRMS) | | | | 1 | 0.36 | >>0.001 |
| α-Rha | Median, RFU | 22,558,850 | 20,449,363 | 23,362,845 | 33,054,943 | 18,692,460 | 10,159,052 |
| | SD | 15,578,868 | 13,709,120 | 16,599,283 | 18,384,279 | 12,845,010 | 9,080,613 |
| | p-value (vs RRMS) | | | | 0.3 | 0.41 | >>0.001 |
| α-GlcNAc | Median, RFU | 17,387,722 | 17,743,149 | 16,637,097 | 13,726,772 | 12,651,926 | 4,129,203 |
| | SD | 10,033,065 | 8,851,812 | 10,762,770 | 9,614,501 | 8,404,338 | 8,529,847 |
| | p-value (vs RRMS) | | | | 0.8 | 0.07 | >>0.001 |

MS, multiple sclerosis;
OAD, other autoimmune diseases;
OND, other neurological diseases;
PPMS, primary progressive multiple sclerosis;
RRMS, relapsing-remitting multiple sclerosis.

What is claimed is:

1. A method of diagnosing multiple sclerosis in a subject, the method comprising
providing a test sample from a subject;
detecting in said test sample an anti-Glc(α1-4) Glc(α) antibody; and
comparing the levels of said antibodies in said test sample to a control sample, wherein said control sample is selected from the group consisting of one or more individuals that have multiple sclerosis symptoms and have a known multiple sclerosis status, and one or more individuals that do not show multiple sclerosis symptoms thereby diagnosing multiple sclerosis in said subject.

2. The method of claim 1, wherein said method further comprises detecting a second antibody selected from the group consisting of an anti-Glc(α) antibody, an anti-Glc(α1-4) Glc (β) antibody, an anti-Glc (β) antibody, an anti-Gal (β) antibody; an anti-Glc (β1-4) Glc (β1-4) Glc (β) antibody, an anti-GlcNAc (β1-4) GlcNAc (α) antibody, an anti-L-Araf (α) antibody, an anti-L-Rha (α) antibody, an anti-Gal (β1-3) [GlcNAc (β1-6)] GalNAc (α) antibody, an anti-Gal (β1-4) GlcNAc (α)antibody, an anti-Gal (β1-3) GalNAc (α) antibody, an anti-Gal (β1-3) GlcNAc (β) antibody, an anti-GlcA (β) antibody, an anti-GlcA (β) antibody, and an anti-Xyl (α) antibody; and
comparing the levels of the second antibody in said test sample to the levels of the second antibody in a control sample, wherein said control sample is selected from the group consisting of one or more individuals that have multiple sclerosis symptoms and have a known multiple sclerosis status, and one or more individuals that do not show multiple sclerosis symptoms,
thereby diagnosing multiple sclerosis in said subject.

3. The method of claim 2, wherein the second antibody is an anti-Glc (α) antibody or an anti-L-Rha (α) antibody.

4. The method of claim 1, wherein said control sample consists essentially of a population of one or more individuals that have multiple sclerosis symptoms with a known multiple sclerosis status.

5. The method of claim 1, wherein said control sample consists essentially of a population of one or more individuals that have an autoimmune disease other than multiple sclerosis.

6. The method of claim 1, wherein said control sample consists essentially of a population of one or more individuals that have a neurological disease other than multiple sclerosis.

7. The method of claim 1, wherein said test sample is a biological fluid.

8. The method of claim 7, wherein said biological fluid is whole blood, serum, plasma, spinal cord fluid, urine, or saliva.

9. The method of claim 1, wherein said biological fluid is serum.

10. The method of claim 1, wherein said subject is a female.

11. The method of claim 1, wherein said subject is a male.

12. The method of claim 1, wherein said at least one antibody is an IgM type antibody.

13. The method of claim 1, wherein said at least one antibody is an IgA type antibody or an IgG type antibody.

14. The method of claim 2, wherein said anti-Glc (α) antibody is an IgM type antibody.

15. The method of claim 1, wherein said anti-Glc (α1-4) Glc (α) antibody is an IgM type antibody.

16. The method of claim 1, wherein said diagnosis is an early diagnosis of multiple sclerosis.

17. The method of claim 1, wherein said control sample is determined using an Expanded Disability Status Scale (EDSS) assessment or a Magnetic Resonance Imaging (MRI) assessment.

18. The method of claim 1, wherein said control sample is determined using an Expanded Disability Status Scale (EDSS) assessment.

19. The method of claim 1, wherein said method comprises detecting at least two of said antibodies.

20. The method of claim 1, wherein said method comprises detecting at least four of said antibodies.

21. The method of claim 1, wherein said method comprises detecting at least six of said antibodies.

22. A method of diagnosing a multiple sclerosis exacerbation in a subject, the method comprising
providing a test sample from a subject;
detecting an anti-Glc (α) IgM type antibody or an anti-Glc (α1-4) Glc (α) IgM type antibody in said test sample; and
comparing the levels of said antibody in said test sample to a control sample, wherein said control sample is derived from one or more individuals whose multiple sclerosis status is known,
thereby diagnosing multiple sclerosis exacerbation in said subject.

23. The method of claim 22, wherein said method comprises
detecting an anti-Glc (α) IgM type antibody in said test sample; and
comparing the levels of said antibody in said test sample to said control sample.

24. The method of claim 22, wherein said method comprises
detecting an anti-Glc (α1-4) Glc (α) α IgM type antibody in said test sample; and
comparing the levels of said antibody in said test sample to said control sample.

25. The method of claim 22, wherein said method comprises
detecting an anti-α-Glucose IgM type antibody and an anti-Glc (α1-4) Glc (α) a IgM type antibody in said test sample; and
comparing the levels of said antibodies in said test sample to said control sample.

26. The method of claim 22, wherein said control sample consists essentially of a population of one or more individuals in remission multiple sclerosis status that do not show symptoms of a multiple sclerosis exacerbation, and a multiple sclerosis exacerbation is diagnosed in said subject if more anti-Glc (α) antibody or anti-Glc (α1-4) Glc (α) antibody is present in said test sample than in said control sample.

27. The method of claim 22, wherein said control sample consists essentially of a population of one or more individuals that their multiple sclerosis status in exacerbation, and show symptoms of a multiple sclerosis exacerbation, and a multiple sclerosis exacerbation is diagnosed in said subject if similar anti-Glc (α) antibody or anti-Glc (α1-4) Glc (α) antibody levels is present in said test sample and in said control sample.

28. The method of claim 22, wherein said test sample is a biological fluid.

29. The method of claim 28, wherein said biological fluid is whole blood, serum, plasma, spinal cord fluid, urine, or saliva.

30. The method of claim 28, wherein said biological fluid is serum.

31. The method of claim 22, wherein said subject is a female.

32. The method of claim 22, wherein said subject is a male.

33. The method of claim 22, wherein said diagnosis is an early diagnosis of multiple sclerosis exacerbation.

34. The method of claim 22, wherein said subject has been treated by subcutaneous administration of interferon beta.

35. The method of claim 22, wherein said subject has been treated by subcutaneous administration of glitamerer acetate.

36. A method for assessing multiple sclerosis disease activity in a subject, the method comprising
providing a test sample from a subject;
determining whether said test sample contains an anti-α Glucose IgM type antibody or an anti-Glc (α1-4) Glc (α) IgM type antibody; and
comparing the level of said at least one antibody in said test sample to a control sample, wherein said control sample is derived from one or more individuals whose multiple sclerosis disease severity is known.
thereby assessing multiple sclerosis activity in said subject.

37. The method of claim 36, wherein said method comprises
detecting an anti-Glc (α) IgM type antibody in said test sample; and
comparing the levels of said antibody in said test sample to said control sample.

38. The method of claim 35, wherein said method comprises
detecting an anti-Glc (α 1-4) Glc (α) IgM type antibody in said test sample; and
comparing the levels of said antibodies in said test sample to said control sample.

39. The method of claim 35, wherein said method comprises
detecting an anti-Glc (α 1-4) Glc (α) IgM type antibody and an anti-Glc (α) IgM type antibody in said test sample; and
comparing the level of said antibodies in said test sample to said control sample.

40. The method of claim 36, wherein said control sample consists essentially of a population of one or more individuals whose multiple sclerosis disease severity is defined by Expanded Disability Status Scale (EDSS), changes in an EDSS score, or a Magnetic Resonance Imaging (MRI) assessment.

41. The method of claim 36, wherein said test sample is a biological fluid.

42. The method of claim 41, wherein said biological fluid is whole blood, serum, plasma, spinal cord fluid, urine, saliva.

43. The method of claim 41, wherein said biological fluid is serum.

44. The method of claim 36, wherein said subject is a female.

45. The method of claim 36, wherein said subject is a male.

46. The method of claim 36, further comprising selecting a therapeutic agent for treating multiple sclerosis, the method comprising
determining whether said test sample contains anti Glucose α antibody; and
selecting a therapeutic agent and dosage regimen based on the relative levels of said antibody in said subject sample and said control sample.

47. The method of claim 46, wherein said method further comprises
determining whether said test sample contains an anti-Glc (α 1-4) Glc (α) antibody; and
comparing the levels of said an anti-Glc (α 1-4) Glc (α) antibody in said test sample to levels of antibody in a control sample consisting essentially of one or more individuals whose multiple sclerosis status is known.

48. A method of determining the prognosis of multiple sclerosis in a subject, the method comprising
providing a test sample from a subject;
detecting in said test sample an anti-Glc (α 1-4) Glc (α) antibody; and
comparing the levels of said antibodies in said test sample to a control sample, wherein said control sample is selected from the group consisting of one or more individuals that have multiple sclerosis symptoms and have a known multiple sclerosis status, and one or more individuals that do not show multiple sclerosis symptoms
thereby determining the prognosis of multiple sclerosis in said subject.

49. The method of claim 48, wherein said method further comprises detecting a second antibody selected from the group consisting of an anti-Glc (α) antibody, an anti-Glc (α 1-4) Glc (β) antibody, an anti-Glc (β) antibody, an anti-Gal (β) antibody; an anti-Glc (β 1-4) Glc (β 1-4) Glc (β) antibody, an anti-GlcNAc (β 1-4) GlcNAc (β) antibody, an anti-L-Araf (α) antibody, an anti-L-Rha (α) antibody, an anti-Gal (β1-3) [GlcNAc (β1-6)] GalNAc (α)antibody, an anti-Gal (β 1-4) GlcNAc (α)antibody, an anti-Gal (β 1-3) GalNAc (α) antibody, an anti-Gal (β 1-3) GlcNAc (β) antibody, an anti-GlcA (β) antibody, an anti-GlcA (β) antibody, and an anti-Xyl (α) antibody; and
comparing the levels of the second antibody in said test sample to the levels of the second antibody in a control sample, wherein said control sample is selected from the group consisting of one or more individuals that have multiple sclerosis symptoms and have a known multiple sclerosis status, and one or more individuals that do not show multiple sclerosis symptoms,
thereby diagnosing multiple sclerosis in said subject.

50. The method of claim 48, wherein said control sample consists essentially of a population of one or more individuals that have multiple sclerosis symptoms with a known multiple sclerosis status.

51. The method of claim 48, wherein said control sample consists essentially of a population of one or more individuals whose MS is known to become more active.

52. The method of claim 48, wherein said control sample consists essentially of a population of one or more individuals whose MS is known to become more active.

53. The method of claim 48, wherein said test sample is a biological fluid.

54. The method of claim 53, wherein said biological fluid is whole blood, serum, plasma, spinal cord fluid, urine, or saliva.

55. The method of claim 53, wherein said biological fluid is serum.

56. The method of claim 48, wherein said subject is a female.

57. The method of claim 48, wherein said subject is a male.

58. The method of claim 48, wherein said at least one antibody is an IgM type antibody.

59. The method of claim 48, wherein said at least one antibody is an IgA type antibody or an IgG type antibody.

60. The method of claim 49, wherein said anti-Glc (α) antibody is an IgM type antibody.

61. The method of claim 48, wherein said anti-Glc (α 1-4) Glc (α) antibody is an IgM type antibody.

62. The method of claim 48, wherein said control sample is determined using an Expanded Disability Status Scale (EDSS) assessment or a Magnetic Resonance Imaging (MRI) assessment.

63. The method of claim 48, wherein said control sample is determined using an Expanded Disability Status Scale (EDSS) assessment.

64. The method of claim 48, wherein said method comprises detecting at least two of said antibodies.

65. The method of claim 48, wherein said method comprises detecting at least four of said antibodies.

66. The method of claim 48, wherein said method comprises detecting at least six of said antibodies.

67. A kit for diagnosing symptoms associated with, determining the prognosis of, or assessing the activity of, multiple sclerosis in subject, the kit comprising:
a first reagent that specifically detects an anti-Glc (α 1-4) Glc (α) antibody;
a second reagent that specifically detects a second antibody selected from the group consisting of an anti-Glc (α) antibody, an anti-Glc (α 1-4) Glc (β) antibody, an anti-Glc (β3 antibody, an anti-Gal (β) antibody; an anti-Glc (β 1-4) Glc (β 1-4) Glc (β) antibody, an anti-GlcNAc (β 1-4) GlcNAc (β) antibody, an anti-L-Araf (α) antibody, an anti-L-Rha (α) antibody, an anti-Gal (β1-3) [GlcNAc (β1-6)] GalNAc (α)antibody, an anti-Gal (β1-4) GlcNAc (α)antibody, an anti-Gal (β 1-3) GalNAc (α) antibody, an anti-Gal (β 1-3) GlcNAc (β) antibody, an anti-GlcA (β) antibody, an anti-GlcA (β) antibody, and an anti-Xyl (α) antibody; and
directions for using said kit.

68. The kit of claim 67, further comprising a reagent that specifically detects an IgM type antibody.

69. A substrate comprising a reagent that detects an antibody specific for Glc(α1-4) Glc (α) linkage.

70. The substrate of claim 69, further comprising a reagent that detects an antibody selected from the group consisting of an anti-Glc (α) antibody, an anti-Glc (α1-4) Glc (α) antibody, an anti-Glc (α 1-4) Glc (β) antibody, an anti-Glc (β) antibody, an anti-Gal (β) antibody; an anti-Glc (β 1-4) Glc (β 1-4) Glc (β)antibody, an anti-GlcNAc (β 1-4) GlcNAc (β)antibody, an anti-L-Araf (α)antibody, an anti-L-Rha (α)antibody, an anti-Gal (β1-3) [GlcNAc (β1-6)] GalNAc (α)antibody, an anti-Gal (β 1-4) GlcNAc (a)antibody, an anti-Gal (β 1-3) GalNAc (α), an anti-Gal (β 1-3) GlcNAc (β), an anti-GlcA (β) antibody, or an anti-GlcA (β) antibody, and an anti-Xyl (α) antibody.

71. The substrate of claim 69, further comprising a regent that detects an anti-Glc (α) antibody.

72. The substrate of claim 69, further comprising a reagent that detects an anti-L-Rha (α) antibody.

73. The substrate of claim 69, further comprising a reagent that detects an anti-L-Rha (α) antibody.

74. The substrate of claim 69, wherein said substrate is planar.

75. The substrate of claim 69, wherein said substrate is provided as a well of a micro-titer plate.

76. The substrate of claim 69, wherein said reagent is a monosaccharide or oligosaccharide.
